(19) United States
(12) Patent Application Publication
(10) Pub. No.: US 2025/0290070 A1
(43) Pub. Date: Sep. 18, 2025

(54) DNAZYME SENSORS AND IMAGING PROBES FOR DETECTION OF METAL IONS AND METABOLITES IN CELLS

(71) Applicant: BOARD OF REGENTS, THE UNIVERSITY OF TEXAS SYSTEM, Austin, TX (US)

(72) Inventors: Yi LU, Austin, TX (US); Quanbing MOU, Austin, TX (US)

(21) Appl. No.: 19/081,649

(22) Filed: Mar. 17, 2025

Related U.S. Application Data
(60) Provisional application No. 63/565,821, filed on Mar. 15, 2024.

(51) Int. Cl. C12N 15/113; C12Q 1/25

(52) U.S. Cl. CPC C12N 15/113; C12Q 1/25; C12N 2310/127; C12N 2310/3513; C12N 2310/3517

(57) ABSTRACT

Methods for spatially identifying metal ions in a cell or tissue sample and compositions therefor.

### CROSS-REFERENCE TO RELATED APPLICATIONS

This application claims the benefit of priority to U.S. Provisional Application No. 63/565,821, filed Mar. 15, 2024, which is incorporated by reference herein in its entirety.

### GOVERNMENT SUPPORT CLAUSE

This invention was made with government support under Grant No. R35 GM141931 awarded by the National Institutes of Health. The Government has certain rights in the invention.

### REFERENCE TO SEQUENCE LISTING

The sequence listing submitted on Mar. 17, 2025, as an .XML file entitled “10046-606US1_ST26.xml” created on Mar. 17, 2025, and having a file size of 43,637 bytes is hereby incorporated by reference pursuant to 37 C.F.R. § 1.52(e)(5).

### BACKGROUND

Metal ions play fundamental roles in biology by serving as essential cofactors in processes such as respiration, growth, gene transcription, enzymatic reactions, cell proliferation and immune function. These essential metals can also be toxic at elevated levels and therefore metal import, trafficking, availability and export must be tightly regulated at the cellular level. Collectively, these processes are referred to as homeostasis. Not surprisingly, altered metal homeostasis is associated with a wide array of primary pathologies and diseases including genetic disorders, degenerative diseases, cancer, and diabetes. Metal homeostasis can also be altered secondary to other diseases and treatments.¹ For example, hemochromatosis can occur due to frequent blood transfusions², and zinc deficiency due to chronic liver disease or intestinal malabsorption³. It is relatively straightforward to determine the total metal content of a cell using techniques such as atomic absorption spectroscopy or inductively coupled plasma mass spectrometry, it is much more challenging to define where metals are located and what chemical form they are in. Yet mapping metals in cellular sub-compartments within the cell is a necessary step in understanding metal homeostasis.¹

Interestingly, several evidences suggest metal ions are unlikely to be evenly distributed throughout a cell. First and foremost, imaging techniques have yielded images of uneven distribution of metals in cells.⁴ Secondly, there is evidence, at least in bacteria, that cells exploit compartmentalization to buffer metal ions at different levels in different locations as one mechanism of ensuring the correct metal is loaded into the correct protein.⁵ The idea that transient changes in metal ion concentrations may lead to the generation of metal ion “signals” represents an exciting paradigm for investigating how cells control levels of metal ions and how metal ions influence cellular function. Exploring these parameters requires analytical tools and techniques to define metal content with high spatial and temporal resolution. This has led to significant advances in recent years in the ability to map metals in cells, including the application of analytical techniques as well as the development of probes.¹

A variety of techniques have been developed and used to visualize specific metal ions in biological samples in a way that preserves the spatial distribution. This can be done on either fixed or live specimens. A suite of analytical techniques capable of defining metal distribution have been applied to fixed biological specimens, including spatially resolved mass spectrometry techniques such as secondary ion mass spectrometry (SIMS), and laser ablation inductively coupled plasma mass spectrometry (LA-ICP-MS), electron spectroscopy imaging (ESI) combined with electron energy loss spectroscopy (EELS), and X-ray fluorescence microscopy (XRFM).¹ Each of these techniques allows researchers to define total metal content with high sensitivity and sub-cellular spatial resolution. However, they do not differentiate between “free” and bound metal ions. Fluorescent sensors based on a variety of platforms (small molecule, genetically encoded, etc.) have been developed for an array of metal ions living cells, tissues, and organisms. Currently the achievable spatial resolutions of standard fluorescence microscopies are still relatively low for precise organelle localization.

Thus, there exists a need for improved spatial visualization of metal ions in biological samples. This is at least partially satisfied by the present disclosure.

### SUMMARY

Disclosed herein is a method of spatial analysis of metal ions in a biological sample using a DNAzyme (also referred to as DNAzyme-PAINT (Points Accumulation for Imaging in Nanoscale Topography)) or an aptamer (also referred to as Aptamer-Paint). One or more types of DNAzymes and/or aptamers, each of which can bind one or more specific metal ions (e.g., Mg²⁺) are introduced into a cell. When the DNAzyme or aptamer binds the specific metal ion and, in particular, is exposed to light, it can bind to an imaging probe. The imaging probe can then be identified using a 2D or 3D cell/tissue imagine technique. The DNAzyme or aptamer can be free-floating in the cell or be anchored to a cell structure (e.g., a microtubule or mitochondrion) by an antibody.

In an aspect, provided is a DNAzyme sensor including: a substrate strand including a cleavage site, wherein the cleavage site is uncleaved when a target molecule is not present; and an enzyme strand at least partially complementary to the substrate strand and including a catalytic loop; wherein the catalytic loop is capable of cleaving the substrate strand at the cleavage site in the presence of the target molecule, wherein said cleavage can provide a detectable signal.

In another aspect, provided is a DNAzyme sensor including: a substrate strand including a cleavage site, wherein the cleavage site is uncleaved when magnesium (Mg²⁺) is not present; and an enzyme strand at least partially complementary to the substrate strand and including a catalytic loop; wherein the catalytic loop is capable of cleaving the substrate strand at the cleavage site in the presence of Mg²⁺, wherein said cleavage can provide a detectable signal; and wherein the catalytic loop includes SEQ ID NO: 13 or a variant thereof.

In yet another aspect, provided is a kit including any of the disclosed DNAzyme sensors that include a docking site and an imaging probe; wherein the imaging probe includes the detectable signal and a sequence at least partially complementary to the docking site; and wherein, upon cleavage of the substrate strand in the presence of the target molecule, the imaging probe can bind to the docking site on the DNAzyme sensor, thereby providing a detectable signal.

In yet still another aspect, provided is a kit including any of the disclosed DNAzyme sensors that bind to Mg²⁺ and include a docking site and an imaging probe; wherein the imaging probe includes the detectable signal and a sequence at least partially complementary to the docking site; and wherein, upon cleavage of the substrate strand in the presence of Mg²⁺, the imaging probe can bind to the docking site on the DNAzyme sensor, thereby providing a detectable signal.

In yet still another aspect, provided is cell including any of the disclosed DNAzyme sensors and/or any of the disclosed imaging probes.

In yet still another aspect, provided is a method for spatially identifying a target molecule in a cell, the method including: a) providing to the cell a DNAzyme sensor including: i) a substrate strand including a cleavage site, wherein the cleavage site is initially uncleaved; ii) an enzyme strand at least partially complementary to the substrate strand and including a catalytic loop; iii) a docking site at least partially complementary to the substrate strand; wherein the catalytic loop is capable of cleaving the substrate strand at the cleavage site in the presence of a target molecule; b) providing to the cell an imaging probe including a detectable signal and a sequence at least partially complementary to the docking site of the DNAzyme sensor, wherein, upon cleavage of the substrate strand in the presence of the target molecule, the imaging probe can bind to the docking site; and c) imaging the cell, thereby allowing for spatial identification of the target molecule.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 is a schematic illustration the synthesis of DNA/DNAzyme-antibody conjugates using SEQ ID NO: 12 as an example.

FIG. 2 is a schematic illustration of the engineered 10-23 caged LNA modified DNAzyme (SEQ ID NO: 9) versus the regular 10-23 DNAzyme (SEQ ID NO: 14). FIG. 2 also shows the comparison of the cleavage activity between 10-23 caged LNA modified DNAzyme and the regular 10-23 DNAzyme by using a 20% denaturing PAGE gel.

FIGS. 3A-3B depict super resolution images of different concentrations of the docking strand in HeLa cells by using fluorogenic DNA-PAINT. FIG. 3A is a schematic illustration of fluorogenic DNA-PAINT of docking strand in HeLa cells. FIG. 3B shows the calculated location of different concentrations of Dock-1-(SS)10 (0.05 nM, 0.5 nM, and 5 nM) in HeLa cells.

FIGS. 4A-4E depict fluorogenic 10-23 LNA-modified DNAzyme-PAINT (SEQ ID NO: 12) for microtubule and mitochondria imaging. FIG. 4A is a schematic illustration of fluorogenic 10-23 LNA-modified DNAzyme-PAINT in the presence of light, magnesium, and imager probe. FIG. 4B is a schematic illustration of microtubule imaging with fluorogenic 10-23 LNA modified DNAzyme-PAINT. FIG. 4C is a schematic illustration of mitochondria imaging with fluorogenic 10-23 LNA-modified DNAzyme-PAINT. FIG. 4D shows microtubules in a single cell after data reconstruction. FIG. 4E shows mitochondria in a single cell after data reconstruction. Scale bar: 5000 nm.

FIGS. 5A-5F depict fluorogenic 10-23 LNA-modified DNAzyme-PAINT (SEQ ID NO: 11) for labile Mg²⁺ imaging. FIG. 5A is a schematic illustration of fluorogenic 10-23 LNA-modified DNAzyme-PAINT for Mg²⁺ imaging by using microtubule as reference. FIG. 5B shows microtubules in a single cell after data reconstruction. FIG. 5C shows labile Mg²⁺ imaging in a single cell after data reconstruction. FIG. 5D is a schematic illustration of fluorogenic 10-23 LNA-modified DNAzyme-PAINT for Mg²⁺ imaging by using mitochondria as reference. FIG. 5E shows mitochondria in a single cell after data reconstruction. FIG. 5F shows labile Mg²⁺ imaging in a single cell after data reconstruction. Scale bar: 5000 nm.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion polynucleotides also are contemplated herein. A “fusion polynucleotide” refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide. A variant polynucleotide may have substantially the same functional activity as a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

The term “mismatched” or “mismatched target sequence” refers to an off-target sequence that is not perfectly complementary to the first DNA sequence or the second DNA sequence of the chimeric deoxyribonucleic acid described herein. The dual retargeted DNA may have at least one mismatch, but can also have 2, 3, 4, 5, 6 or 7 or more mismatched nucleotides to the off-target sequence.

As used herein, the term “at least partially complementary” refers to two nucleic acid sequences which are partially or fully complementary. For example, two sequences may be “at least partially complementary” if at least about 50% (e.g., at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, about 100%) of one sequence is complementary to at least about 50% (e.g., at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, about 100%) of the other sequence.

As used herein, the term “detecting” used in context of detecting a signal from a detectable label or detectable signal to indicate the presence of a target nucleic acid (such as a signaling nucleic acid) in the sample does not require the method to provide 100% sensitivity and/or 100% specificity. As is well known, “sensitivity” is the probability that a test is positive, given that the sample has a target nucleic acid sequence, while “specificity” is the probability that a test is negative, given that the sample does not have the target nucleic acid sequence. A sensitivity of at least 50% is preferred, although sensitivities of at least 60%, at least 70%, at least 80%, at least 90% and at least 99% are clearly more preferred. A specificity of at least 50% is preferred, although sensitivities of at least 60%, at least 70%, at least 80%, at least 90% and at least 99% are clearly more preferred. Detecting also encompasses assays with false positives and false negatives. False negative rates may be 1%, 5%, 10%, 15%, 20% or even higher. False positive rates may be 1%, 5%, 10%, 15%, 20% or even higher. The term “detecting” is also used in the context of detecting the amplified target nucleic acid by its melting temperature using melting curve analysis, as is known in the art.

As used herein, “detectable labels” or “detectable signals” are chemical or biochemical moieties useful for labeling a nucleic acid (including a single nucleotide), amino acid, or antibody. “Detectable labels” or “detectable signals” include fluorescent agents, chemiluminescent agents, chromogenic agents, quenching agents, radionuclides, enzymes, substrates, cofactors, inhibitors, magnetic particles, quantum dots, and other moieties known in the art. “Detectable labels” or “detectable signals” are capable of generating a measurable signal, or can be used to capture nucleic acids, and may be covalently or noncovalently joined to an oligonucleotide or nucleotide (e.g., a non-natural nucleotide).

A detectable label or detectable signal is said to be “activated” when it is completely or partially activated (i.e., detectable). For example, a detectable label or detectable signal can be said to be “activated” when the intensity of its measurable signal is greater than about 50% (e.g., greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98%, greater than about 99%, about 100%) of the expected intensity of its measurable signal based on a reasonable assumption by one of skill in the art. It is understood that the threshold to classify a detectable label or detectable signal as “activated” can be reasonably ascertained by one of skill in the art depending on the application and properties (e.g., sensitivity, specificity, etc.) of the detectable label or detectable signal.

A detectable label or detectable signal is said to be “deactivated” when it is completely or partially deactivated (i.e., not detectable). For example, a detectable label or detectable signal can be said to be “deactivated” when the intensity of its measurable signal is less than about 50% (e.g., less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, less than about 1%, about 0%) of the expected intensity of its measurable signal based on a reasonable assumption by one of skill in the art. It is understood that the threshold to classify a detectable label or detectable signal as “deactivated” can be reasonably ascertained by one of skill in the art depending on the application and properties (e.g., sensitivity, specificity, etc.) of the detectable label or detectable signal.

As used herein, the term “DNAzymes,” also called “deoxyribozymes,” are DNA molecules that display enzymatic activities, such as protein enzymes and ribozymes, in the presence of a cofactor such as metal ions or another target molecule.

### DNAzyme Sensors

In an aspect, provided is a DNAzyme sensor including: a substrate strand including a cleavage site, wherein the cleavage site is uncleaved when a target molecule is not present; and an enzyme strand at least partially complementary to the substrate strand and including a catalytic loop; wherein the catalytic loop is capable of cleaving the substrate strand at the cleavage site in the presence of the target molecule, wherein said cleavage can provide a detectable signal. In some aspects, the target molecule can be a metal ion. For example, in some aspects, the metal ion can be selected from Li⁺, Na⁺, K⁺, Mg²⁺, Mn²⁺, Zn²⁺, UO₂²⁺, Fe²⁺, Fe³⁺, Cu⁺, or Cu²⁺. In other aspects, the target molecule can be a metabolite.

In another aspect, provided is a DNAzyme sensor including: a substrate strand including a cleavage site, wherein the cleavage site is uncleaved when magnesium (Mg²⁺) is not present; and an enzyme strand at least partially complementary to the substrate strand and including a catalytic loop; wherein the catalytic loop is capable of cleaving the substrate strand at the cleavage site in the presence of Mg²⁺, wherein said cleavage can provide a detectable signal; and wherein the catalytic loop includes SEQ ID NO: 13 (GGC TAG CTA CAA CGA) or a variant thereof.

In some aspects, the cleavage site can include at least one RNA base. For example, in some such aspects, the cleavage site can include 1 RNA base, 2 RNA bases, 3 RNA bases, 4 RNA bases, or 5 RNA bases. In some such aspects, the cleavage site can include from 1 RNA base to 5 RNA bases (e.g., from 2 RNA bases to 5 RNA bases, from 3 RNA bases to 5 RNA bases, from 4 RNA bases to 5 RNA bases, from 1 RNA base to 4 RNA bases, from 2 RNA bases to 4 RNA bases, from 3 RNA bases to 4 RNA bases, from 1 RNA base to 3 RNA bases, from 2 RNA bases to 3 RNA bases, from 1 RNA base to 2 RNA bases). In other such aspects, the cleavage site can include more than 5 RNA bases. In some aspects, when more than one RNA base is present, all RNA bases may be immediately adjacent. In other aspects, when more than one RNA base is present, at least one RNA base may be distanced from the other RNA bases by at least one other nucleotide.

In some aspects, the cleavage site can be interspersed between two segments of nucleic acid. In some such aspects, the two segments of nucleic acid can have a same length. In other such aspects, the two segments of nucleic acid can have different lengths.

In some aspects, the substrate strand and/or the enzyme strand can include DNA. In some aspects, the substrate strand can further include at least one non-natural nucleic acid. For example, in some such aspects, the at least one non-natural nucleic acid can be a locked nucleic acid (LNA) or a 2′-fluoro arabino nucleic acid (FANA). In some aspects, the substrate strand and/or the enzyme strand can include a terminal dithiol, thiosulfonate, benzopolysulfane, or another sulfur-containing ring structure.

In some aspects, the substrate strand and the enzyme strand can be linked together. In some such aspects, the substrate strand and the enzyme strand can be directly linked together. In other such aspects, the substrate strand and the enzyme strand can be linked together by a linker.

In some aspects, the linker can include nucleic acid, peptide, polymer, or any combination thereof. For example, in some aspects, the linker can include at least 1 nucleotide (e.g., at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides). In some aspects, the linker can include up to 10 nucleotides (e.g., up to 9 nucleotides, up to 8 nucleotides, up to 7 nucleotides, up to 6 nucleotides, up to 5 nucleotides, up to 4 nucleotides, up to 3 nucleotides, up to 2 nucleotides, up to 1 nucleotide).

It is considered that the linker can include any number of nucleotides ranging from any of the minimum values described above to any of the maximum values described above. For example, in some aspects, the linker can include from 1 nucleotide to 10 nucleotides (e.g., from 2 nucleotides to 9 nucleotides, from 3 nucleotides to 8 nucleotides, from 4 nucleotides to 7 nucleotides, from 5 nucleotides to 6 nucleotides, from 1 nucleotide to 6 nucleotides, from 2 nucleotides to 5 nucleotides, from 3 nucleotides to 4 nucleotides, from 5 nucleotides to 10 nucleotides, from 6 nucleotides to 9 nucleotides, from 7 nucleotides to 8 nucleotides.)

In some aspects, the DNAzyme sensor can include about 80% similarity or more (e.g., about 81% similarity or more, about 82% similarity or more, about 83% similarity or more, about 84% similarity or more, about 85% similarity or more, about 86% similarity or more, about 87% similarity or more, about 88% similarity or more, about 89% similarity or more, about 90% similarity or more, about 91% similarity or more, about 92% similarity or more, about 93% similarity or more, about 94% similarity or more, about 95% similarity or more, about 96% similarity or more, about 97% similarity or more, about 98% similarity or more, about 99% similarity or more, about 100% similarity) to any one of SEQ ID NOs: 5-9 or SEQ ID NOs: 11-12. In some aspects, the DNAzyme sensor can include any one of SEQ ID NOs: 5-9 or SEQ ID NOs: 11-12. In some aspects, the DNAzyme sensor can consist of any one of SEQ ID NOs: 5-9 or SEQ ID NOs: 11-12.

In some aspects, the DNAzyme sensor can further include an antibody conjugated to the DNAzyme sensor. In some aspects, the antibody can be conjugated to the 5′ end or the 3′ end of the enzyme strand. In other aspects, the antibody can be conjugated to the 5′ end or the 3′ end of the substrate stand. In yet other aspects, the antibody can be conjugated to both the substrate strand and the enzyme strand.

In some aspects, the antibody can bind to a structure within a cell. For example, in some aspects, the antibody can bind to a microtubule, a mitochondrion, endoplasmic reticulum, Golgi apparatus, or any other organelle or subcellular structure. In some aspects, the antibody can be a primary antibody. In other aspects, the antibody can be a secondary antibody.

In some aspects, the detectable signal can be a fluorophore or fluorescent dye. In some such aspects, the fluorophore or fluorescent dye can be, but is not limited to Hydroxycoumarin, Alexa fluor, Aminocoumarin, Methoxycoumarin, Cascade Blue, Pacific Blue, Pacific Orange, Lucifer yellow, Alexa fluor 430, NBD, R-Phycoerythrin (PE), PE-Cy5 conjugates, PE-Cy7 conjugates, Red 613, PerCP, Cy2, TruRed, FluorX, Fluorescein, FAM, BODIPY-FL, TET, Alexa fluor 532, HEX, TRITC, Cy3, TMR, Alexa fluor 546, Alexa fluor 555, Tamara, X-Rhodamine, Lissamine Rhodamine B, ROX, Alexa fluor 568, Cy3.5 581, Texas Red, Alexa fluor 594, Alexa fluor 633, LC red 640, Allophycocyanin (APC), Alexa fluor 633, APC-Cy7 conjugates, Cy5, Cy5.5, LC red 705, Cy7, IRDye 800 CW, IRDye 700, Cy7.5, Dy780, Dy781, DyLight 800, IRDye 800 CW, Alexa Fluor 647, Alexa Fluor 488, Alexa Fluor 660, Alexa Fluor 680, Alexa Fluor 750, Alexa Fluor 790, JOE, or MAX.

In some aspects, the detectable signal can be a photoacoustic dye; and wherein, when the substrate strand is cleaved, the detectable signal can be activated upon exposure to an acoustic signal. In some such aspects, the detectable signal can be indocyanine green, methylene blue, Evans blue, trypan blue, patent blue, IRDye800CW, DiR, Cy7, Cy7.5, or porphyrin.

In some aspects, the detectable signal can be conjugated to a first end of the substrate strand, and a quencher can be conjugated to a complementary end of the enzyme strand. In other aspects, the detectable signal can be conjugated to a first end of the substrate strand, and a quencher can be conjugated to a second end of the substrate strand. In yet other aspects, the detectable signal can be conjugated to a first end of the substrate strand, a first quencher can be conjugated to a complementary end of the enzyme strand, and a second quencher can be conjugated to a second end of the substrate strand.

In some aspects, the quencher can be, but is not limited to, DQ-I, Dabcyl, Eclipse, Iowa Black FQ, BHQ-1, QSY-7, BHQ-2, DDQ-II, Iowa Black RQ, QSY-21, BHQ-3, IRDye QC-1, or ZEN. In some aspects, when more than one quencher is present, each quencher can be the same or different.

In some aspects, the catalytic loop can have a dissociation constant (K_{d}) of Mg²⁺ of about 30 nM or more (e.g., about 30.5 nM or more, about 31 nM or more, about 31.5 nM or more, about 32 nM or more, about 32.5 nM or more, about 33 nM or more, about 33.5 nM or more, about 34 nM or more, about 34.5 nM or more, about 35 nM or more, about 35.5 nM or more, about 36 nM or more, about 36.5 nM or more, about 37 nM or more, about 37.5 nM or more, about 38 nM or more, about 38.5 nM or more, about 39 nM or more, about 39.5 nM or more, about 40 nM or more). In some aspects, the catalytic loop can have a dissociation constant (K_{d}) of Mg²⁺ of about 40 nM or less (e.g., about 39.5 nM or less, about 39 nM or less, about 38.5 nM or less, about 38 nM or less, about 37.5 nM or less, about 37 nM or less, about 36.5 nM or less, about 36 nM or less, about 35.5 nM or less, about 35 nM or less, about 34.5 nM or less, about 34 nM or less, about 33.5 nM or less, about 33 nM or less, about 32.5 nM or less, about 32 nM or less, about 31.5 nM or less, about 31 nM or less, about 30.5 nM or less, about 30 nM or less). The catalytic loop can have a dissociation constant (K_{d}) of Mg²⁺ ranging from any of the minimum values described above to any of the maximum values described above. For example, in some aspects, the catalytic loop can have a dissociation constant (K_{d}) of Mg²⁺ of from about 30 nM to about 40 nM (e.g., from about 30.5 nM to about 39.5 nM, from about 31 nM to about 39 nM, from about 31.5 nM to about 38.5 nM, from about 32 nM to about 38 nM, from about 32.5 nM to about 37.5 nM, from about 33 nM to about 37 nM, from about 33.5 nM to about 36.5 nM, from about 34 nM to about 36 nM, from about 34.5 nM to about 35.5 nM, from about 30 nM to about 35 nM, from about 30.5 nM to about 34.5 nM, from about 31 nM to about 34 nM, from about 31.5 nM to about 33.5 nM, from about 32 nM to about 33 nM, from about 35 nM to about 40 nM, from about 35.5 nM to about 39.5 nM, from about 36 nM to about 39 nM, from about 36.5 nM to about 38.5 nM, from about 37 nM to about 38 nM).

In some aspects, the DNAzyme sensor can include a docking site that is at least partially complementary to the substrate strand; and, upon cleavage of the substrate strand, the docking site may be at least partially exposed. For example, in some aspects, cleavage of the substrate strand can decrease the melting temperature of the DNAzyme sensor, thereby causing the cleaved portion of the substrate strand to at least partially disassociate from the docking site.

In yet another aspect, provided is a kit including any of the disclosed DNAzyme sensors that include a docking site and an imaging probe; wherein the imaging probe includes the detectable signal and a sequence at least partially complementary to the docking site; and wherein, upon cleavage of the substrate strand in the presence of the target molecule, the imaging probe can bind to the docking site on the DNAzyme sensor, thereby providing a detectable signal. In some aspects, the target molecule can be a metal ion. For example, in some aspects, the metal ion can be selected from Li⁺, Na⁺, K⁺, Mg²⁺, Mn²⁺, Zn²⁺, UO₂²⁺, Fe²⁺, Fe³⁺, Cu⁺, or Cu²⁺. In other aspects, the target molecule can be a metabolite.

In yet still another aspect, provided is a kit including any of the disclosed DNAzyme sensors that bind to Mg²⁺ and include a docking site and an imaging probe; wherein the imaging probe includes the detectable signal and a sequence at least partially complementary to the docking site; and wherein, upon cleavage of the substrate strand in the presence of Mg²⁺, the imaging probe can bind to the docking site on the DNAzyme sensor, thereby providing a detectable signal.

In some aspects, the detectable signal can be a fluorophore or a fluorescent dye conjugated to the imaging probe, for example, any of the fluorophores or fluorescent dyes described above. In some aspects, the detectable signal can be a photoacoustic dye conjugated to the imaging probe, for example, any of the photoacoustic dyes described above; and wherein, when the substrate strand is cleaved, the detectable signal can be activated upon exposure to an acoustic signal.

In some aspects, the imaging probe can include about 80% similarity or more (e.g., about 81% similarity or more, about 82% similarity or more, about 83% similarity or more, about 84% similarity or more, about 85% similarity or more, about 86% similarity or more, about 87% similarity or more, about 88% similarity or more, about 89% similarity or more, about 90% similarity or more, about 91% similarity or more, about 92% similarity or more, about 93% similarity or more, about 94% similarity or more, about 95% similarity or more, about 96% similarity or more, about 97% similarity or more, about 98% similarity or more, about 99% similarity or more, about 100% similarity) to any one of SEQ ID NOs: 2-4. In some aspects, the imaging probe can include any one of SEQ ID NOs: 2-4. In some aspects, the imaging probe can consist of any one of SEQ ID NOs: 2-4.

In yet still another aspect, provided is cell including any of the disclosed DNAzyme sensors and/or any of the disclosed imaging probes.

### Methods

In an aspect, provided is a method for spatially identifying a target molecule in a cell, the method including: a) providing to the cell a DNAzyme sensor including: i) a substrate strand including a cleavage site, wherein the cleavage site is initially uncleaved; ii) an enzyme strand at least partially complementary to the substrate strand and including a catalytic loop; iii) a docking site at least partially complementary to the substrate strand; wherein the catalytic loop is capable of cleaving the substrate strand at the cleavage site in the presence of a target molecule; b) providing to the cell an imaging probe including a detectable signal and a sequence at least partially complementary to the docking site of the DNAzyme sensor, wherein, upon cleavage of the substrate strand in the presence of the target molecule, the imaging probe can bind to the docking site; and c) imaging the cell, thereby allowing for spatial identification of the target molecule.

In some aspects, the DNAzyme sensor can include any of the DNAzyme sensors described above. In some aspects, the imaging probe can include any of the imaging probes described above. In some aspects, the method can use any of the kits described above.

In some aspects, the target molecule can be a metal ion. For example, in some aspects, the metal ion can be selected from Li⁺, Na⁺, K⁺, Mg²⁺, Mn²⁺, Zn²⁺, UO₂²⁺, Fe²⁺, Fe³⁺, Cu⁺, or Cu²⁺. In other aspects, the target molecule can be a metabolite.

In some aspects, the DNAzyme sensor can further include an antibody conjugated to the DNAzyme sensor. In some aspects, the antibody can be conjugated to the 5′ end or the 3′ end of the enzyme strand. In other aspects, the antibody can be conjugated to the 5′ end or the 3′ end of the substrate stand. In yet other aspects, the antibody can be conjugated to both the substrate strand and the enzyme strand.

In some aspects, the antibody can bind to a structure within a cell. For example, in some aspects, the antibody can bind to a microtubule, a mitochondrion, endoplasmic reticulum, Golgi apparatus, or any other organelle or subcellular structure. In some aspects, the antibody can be a primary antibody. In other aspects, the antibody can be a secondary antibody. In some such aspects, the secondary antibody can bind to a naturally occurring antibody present on the structure within the cell. In other such aspects, the method can include, before step a), providing to the cell a primary antibody that can bind to the structure within the cell.

In some aspects, step a) can further include irradiating the cell with a light source to activate cleavage of the substrate strand. In some aspects, the light source can be visible light. In some aspects, the light source can have a wavelength of about 350 nm or more (e.g., about 360 nm or more, about 370 nm or more, about 380 nm or more, about 390 nm or more, about 400 nm or more, about 410 nm or more, about 420 nm or more, about 430 nm or more, about 440 nm or more, about 450 nm or more, about 460 nm or more, about 470 nm or more, about 480 nm or more, about 490 nm or more, about 500 nm or more). In some aspects, the light source can have a wavelength of about 500 nm or less (e.g., about 490 nm or less, about 480 nm or less, about 470 nm or less, about 460 nm or less, about 450 nm or less, about 440 nm or less, about 430 nm or less, about 420 nm or less, about 410 nm or less, about 400 nm or less, about 390 nm or less, about 380 nm or less, about 370 nm or less, about 360 nm or less, about 350 nm or less). The light source can have a wavelength ranging from any of the minimum values described above to any of the maximum values described above. For example, in some aspects, the light source can have a wavelength of from about 350 nm to about 500 nm (e.g., from about 360 nm to about 490 nm, from about 370 nm to about 480 nm, from about 380 nm to about 470 nm, from about 390 nm to about 460 nm, from about 400 nm to about 450 nm, from about 410 nm to about 440 nm, from about 420 nm to about 430 nm, from about 350 nm to about 430 nm, from about 360 nm to about 420 nm, from about 370 nm to about 410 nm, from about 380 nm to about 400 nm, from about 420 nm to about 500 nm, from about 430 nm to about 490 nm, from about 440 nm to about 480 nm, from about 450 nm to about 470 nm).

In some aspects, the cell can be irradiated for about 10 seconds or more (e.g., about 15 seconds or more, about 20 seconds or more, about 25 seconds or more, about 30 seconds or more, about 35 seconds or more, about 40 seconds or more, about 45 seconds or more, about 50 seconds or more, about 55 seconds or more, about 60 seconds or more, about 65 seconds or more, about 70 seconds or more, about 75 seconds or more, about 80 seconds or more, about 85 seconds or more, about 90 seconds or more, about 95 seconds or more, about 100 seconds or more, about 105 seconds or more, about 110 seconds or more, about 115 seconds or more, about 120 seconds or more). In some aspects, the cell can be irradiated for about 120 seconds or less (e.g., about 115 seconds or less, about 110 seconds or less, about 105 seconds or less, about 100 seconds or less, about 95 seconds or less, about 90 seconds or less, about 85 seconds or less, about 80 seconds or less, about 75 seconds or less, about 70 seconds or less, about 65 seconds or less, about 60 seconds or less, about 55 seconds or less, about 50 seconds or less, about 45 seconds or less, about 40 seconds or less, about 35 seconds or less, about 30 seconds or less, about 25 seconds or less, about 20 seconds or less, about 15 seconds or less, about 10 seconds or less). The cell can be irradiated for a duration ranging from any of the minimum values described above to any of the maximum values described above. For example, in some aspects, the cell can be irradiated for from about 10 seconds to about 120 seconds (e.g., from about 15 seconds to about 115 seconds, from about 20 seconds to about 110 seconds, from about 25 seconds to about 105 seconds, from about 30 seconds to about 100 seconds, from about 35 seconds to about 95 seconds, from about 40 seconds to about 90 seconds, from about 45 seconds to about 85 seconds, from about 50 seconds to about 80 seconds, from about 55 seconds to about 75 seconds, from about 60 seconds to about 70 seconds, from about 10 seconds to about 65 seconds, from about 15 seconds to about 60 seconds, from about 20 seconds to about 55 seconds, from about 25 seconds to about 50 seconds, from about 30 seconds to about 45 seconds, from about 35 seconds to about 40 seconds, from about 65 seconds to about 120 seconds, from about 70 seconds to about 115 seconds, from about 75 seconds to about 110 seconds, from about 80 seconds to about 105 seconds, from about 85 seconds to about 100 seconds, from about 90 seconds to about 95 seconds).

In some aspects, steps a) and b) can be carried out simultaneously. In other aspects, step a) can be carried out before step b). For example, in some aspects, step a) can further include incubating the cell with the DNAzyme sensor to allow the cell to uptake the DNAzyme sensor prior to step b). In some aspects, the cell can be incubated with the DNAzyme sensor for about 30 minutes or more (e.g., about 45 minutes or more, about 1 hour or more, about 2 hours or more, about 3 hours or more, about 4 hours or more, about 5 hours or more, about 6 hours or more, about 8 hours or more, about 12 hours or more, about 18 hours or more, about 24 hours or more, about 36 hours or more, about 48 hours or more, about 60 hours or more, about 72 hours or more). In some aspects, the cell can be incubated with the DNAzyme sensor for about 72 hours or less (e.g., about 60 hours or less, about 48 hours or less, about 36 hours or less, about 24 hours or less, about 18 hours or less, about 12 hours or less, about 8 hours or less, about 6 hours or less, about 5 hours or less, about 4 hours or less, about 3 hours or less, about 2 hours or less, about 1 hour or less, about 45 minutes or less, about 30 minutes or less). The cell can be incubated with the DNAzyme sensor for a duration ranging from any of the minimum values described above to any of the maximum values described above. For example, in some aspects, the cell can be incubated with the DNAzyme sensor for from about 30 minutes to about 72 hours (e.g., from about 45 minutes to about 60 hours, from about 1 hour to about 48 hours, from about 2 hours to about 36 hours, from about 3 hours to about 24 hours, from about 4 hours to about 18 hours, from about 5 hours to about 12 hours, from about 6 hours to about 8 hours, from about 12 hours to about 5 hours, from about 18 hours to about 4 hours, from about 24 hours to about 3 hours, from about 36 hours to about 2 hours, from about 48 hours to about 1 hour, from about 60 hours to about 45 minutes, from about 72 hours to about 30 minutes, from about 30 minutes to about 8 hours, from about 45 minutes to about 6 hours, from about 1 hour to about 5 hours, from about 2 hours to about 4 hours, from about 6 hours to about 72 hours, from about 12 hours to about 48 hours, from about 18 hours to about 36 hours, from about 60 hours to about 8 hours).

In some aspects, the DNAzyme sensor and the imaging probe can be provided in a ratio of about 1:1 or more (e.g., about 2:1 or more, about 3:1 or more, about 4:1 or more, about 5:1 or more, about 10:1 or more, about 15:1 or more, about 20:1 or more, about 25:1 or more, about 30:1 or more, about 35:1 or more, about 40:1 or more, about 45:1 or more, about 50:1 or more, about 55:1 or more, about 60:1 or more, about 65:1 or more, about 70:1 or more, about 75:1 or more, about 80:1 or more, about 85:1 or more, about 90:1 or more, about 95:1 or more, about 100:1 or more). In some aspects, the DNAzyme sensor and the imaging probe can be provided in a ratio of about 100:1 or less (e.g., about 95:1 or less, about 90:1 or less, about 85:1 or less, about 80:1 or less, about 75:1 or less, about 70:1 or less, about 65:1 or less, about 60:1 or less, about 55:1 or less, about 50:1 or less, about 45:1 or less, about 40:1 or less, about 35:1 or less, about 30:1 or less, about 25:1 or less, about 20:1 or less, about 15:1 or less, about 10:1 or less, about 5:1 or less, about 4:1 or less, about 3:1 or less, about 2:1 or less, about 1:1 or less). The DNAzyme sensor and the imaging probe can be provided in a ratio ranging from any of the minimum values described above to any of the maximum values described above. For example, in some aspects, the DNAzyme sensor and the imaging probe can be provided in a ratio of from about 1:1 to about 100:1 (e.g., from about 2:1 to about 95:1, from about 3:1 to about 90:1, from about 4:1 to about 85:1, from about 5:1 to about 80:1, from about 10:1 to about 75:1, from about 15:1 to about 70:1, from about 20:1 to about 65:1, from about 25:1 to about 60:1, from about 30:1 to about 55:1, from about 35:1 to about 50:1, from about 40:1 to about 45:1, from about 1:1 to about 45:1, from about 2:1 to about 40:1, from about 3:1 to about 35:1, from about 4:1 to about 30:1, from about 5:1 to about 25:1, from about 10:1 to about 20:1, from about 40:1 to about 100:1, from about 45:1 to about 95:1, from about 50:1 to about 90:1, from about 55:1 to about 85:1, from about 60:1 to about 80:1, from about 65:1 to about 75:1).

In some aspects, step a) can further include providing to the cell two or more DNAzyme sensors, wherein the catalytic loop of each DNAzyme sensor is capable of cleaving each substrate strand at its cleavage site in the presence of a different target molecule; and the method can be used to multiplex more than one target molecule at a time. In some aspects, at least one target molecule can be a metal ion. In some aspects, at least one target molecule can be a metal ion and at least another target molecule may not be a metal ion. In other aspects, two different metal ions can be detected simultaneously. In some aspects, at least one target molecule can be Mg²⁺.

In some aspects, the DNAzyme sensors can have the same docking sites. In other aspects, the DNAzyme sensors can have different docking sites, and step a) can further include providing to the cell two or more imaging probes, wherein each imaging probe can include a sequence at least partially complementary to the docking site of a different DNAzyme sensor. In some aspects, each imaging probe can include the same or different detectable signal.

In some aspects, the method can further include, after step b) and before step c), washing the cell with a buffer.

In some aspects, the cell can be within a tissue. In some aspects, the cell can be mammalian. For example, in some such aspects, the cell can be human. In other aspects, the cell can be a plant cell.

In some aspects, the cell can be imaged using 3D imaging. In some aspects, the imaging can be done using a super-resolution microscope. For example, in some such aspects, the microscope can include stimulated emission depletion (STED) microscopy, structured illumination microscopy (SIM), and stochastic optical reconstruction microscopy (STORM)/photoactivation localization microscopy (PALM).

In some aspects, the method can be used to quantify the target molecule in the cell. In some such aspects, software can be used to analyze imaging data to quantify the target molecule in the cell. For example, in some aspects, the software can be Picasso.

In some aspects, the method can be carried out in vitro, ex vivo, or in vivo.

### EXAMPLES

### Example 1: DNAzyme-PAINT and Aptamer-PAINT: 3D Super-Resolution Imaging of Metal Ions and Metabolites in Cells

Metal ions and metabolites are crucial in biological processes, prompting the development of various techniques to visualize them in biological samples. However, the spatial resolution offered by standard technologies often falls short of providing precise organelle localization, limiting understanding of metal homeostasis, particularly regarding metabolites and labile metal ions. The advent of super-resolution fluorescence microscopy has opened new avenues for sensing metabolites and metal ions. In this context, disclosed herein is the DNAzyme-PAINT and aptamer-PAINT techniques for super-resolution imaging of labile metal ions and metabolites. These methods combine DNA-PAINT's imaging capabilities with the selective binding of the catalytic activity of DNAzymes or finding ability of DNA aptamers. Utilizing DNAzyme-PAINT and aptamer-PAINT enables us to depict the 3D super-resolution spatial distribution of metal ions and metabolites within cells, thereby enhancing comprehension of their essential roles and dynamics.

DNAzyme-PAINT and DNA Aptamer-PAINT enable the visualization of metal ions and metabolites within cells at a 3D super-resolution level. These technologies are versatile, allowing for the imaging of various metabolites and metal ions through simple swaps of DNAzymes and aptamers. Distinct from DNA-PAINT, which is limited to imaging subcellular organelles or biomacromolecules, DNAzyme-PAINT and Aptamer-PAINT offer a significant advancement. Traditional biosensors for metal ions and metabolites imaging only offer a broad distribution overview, lacking the super-resolution detail provided by these innovative techniques.

DNAzyme-PAINT offers a groundbreaking tool for capturing labile metal ions at a super-resolution scale in a single cell, while the DNA Aptamer-PAINT introduces an approach for 3D super-resolution imaging of metabolites within individual cells.

Spatially resolved mass spectrometry techniques, including secondary ion mass spectrometry (SIMS), laser ablation inductively coupled plasma mass spectrometry (LA-ICP-MS), electron spectroscopy imaging coupled with electron energy loss spectroscopy (EELS), and X-ray fluorescence microscopy, enable researchers to accurately measure total metal content with high sensitivity and sub-cellular precision. However, these methods cannot distinguish between labile and bound metal ions. To address this, fluorescent sensors across various platforms (small molecules, genetically encoded, etc.) have been developed for detecting metal ions in living cells, tissues, and organisms. Yet, the spatial resolution of conventional fluorescence microscopy remains limited for precise organelle mapping. DNAzyme-PAINT, in contrast, offers 3D super-resolution imaging of labile metal ions within individual cells.

Similarly, while mass spectroscopy stands as the cutting-edge technology for metabolite analysis, it falls short in providing spatial information. Biosensors for metabolites encounter the same limitations as those for metal ions. Aptamer-PAINT, however, offers a solution by enabling 3D super-resolution imaging of metabolites within single cells, surpassing the spatial resolution limitations of traditional methods.

Both aptamer-PAINT and DNAzyme-PAINT could be realized in plants.

While spatial genomics and proteomics have significantly advanced biology and medicine, adapting their methodologies for visualizing metabolites and metal ions proves challenging. This difficulty arises because metabolites and metal ions are small, structurally diverse, and highly mobile within biological systems, making it challenging to selectively probe them with high spatiotemporal resolution. DNA-based Points Accumulation for Imaging in Nanoscale Topography (DNA-PAINT) microscopy has gained popularity for its ability to produce high-quality images of subcellular organelles and proteins with relative ease. However, to date, no biosensing system has been capable of providing 3D super-resolution spatial information on metabolites and labile metal ions within cells. DNAzyme-PAINT and DNA aptamer-PAINT techniques bridge this gap, offering unprecedented insight into the spatial distribution of metabolites and labile metal ions within individual cells. This approach enhances understanding of cellular homeostasis involving metabolites and labile metal ions.

This approach combines DNAzyme or aptamer technology with DNA-PAINT to map the 3D super-resolution spatial distribution of metabolites and labile metal ions within cells. Upon target recognition by the DNAzyme or aptamer, a docking strand is exposed, dynamically interacting with the imager strand used in DNA-PAINT. This process translates the spatial presence of metabolites and labile metal ions into that of docking strands. Consequently, this method utilizes conventional DNA-PAINT technology to deliver detailed 3D super-resolution spatial information on metabolites and labile metal ions.

These techniques furnish detailed 3D super-resolution insights into the spatial distribution of metabolites and labile metal ions. This allows us to correlate specific location data with corresponding subcellular organelle information, enhancing understanding of cellular processes. Gaining a more profound comprehension of the homeostasis of metabolites and labile metal ions within cells can uncover therapeutic targets for diseases linked to their dysregulation, including cancer, diabetes, and more.

### Example 2: Fluorogenic LNA-Modified DNAzyme PAINT: 3D Super-Resolution Imaging of Mg²⁺ in Cells

Metal ions play fundamental roles in biology. A variety of techniques have been developed and used to visualize specific metal ions in biological samples. However, understanding metal homeostasis especially for the labile metal ion is still limited because the achievable spatial resolutions of standard technologies are still relatively low for precise organelle localization. Recent development of super-resolution fluorescence microscopies has created unprecedented new possibilities for metal ion sensing. Herein, a study was conducted which developed the DNAzyme-PAINT technique for super-resolution imaging labile metal ions by combining DNA-PAINT with the cleavage catalytic activity of DNAzyme. With the DNAzyme-PAINT, the 3D distribution of metal ions inside cells would be illustrated, which could promote the understanding of fundamental importance of labile pool metal ions.

### INTRODUCTION

Recent development of super-resolution fluorescence microscopies has created unprecedented new possibilities for fluorescent sensors. DNA-based points accumulation for imaging in nanoscale topography (DNA-PAINT) microscopy is becoming increasingly popular because of the high-quality images that can be obtained with relative ease.⁶ DNA-PAINT⁷⁻¹⁰, in contrast to the other techniques, does not rely on light to switch fluorescent molecules between ‘visible’ and ‘invisible’ states. Instead, it takes advantage of transient DNA binding to achieve the same switching or ‘blinking’ effect. Single-stranded DNA (ssDNA) ‘docking strands’ attached to a biological structure of interest are individually highlight ted as they capture an ‘imager probe’, a complementary ssDNA molecule linked to a fluorophore, from a diffusing pool. High background fluorescence therefore remains a substantial problem in DNA-PAINT and severely limits its application. To overcome those challenges, new probes were engineered to improved ˜26-fold increase in imaging speed with fluorogenic DNA-PAINT.¹¹ Till now, no biosensing system has been reported to analyze metal ion in cells.

Herein, we'll take advantage the metal ion based catalytic activities of DNAzymes to image the labile metal ions by combining DNA-PAINT, denoted as DNAzyme-PAINT. We'll first deliver DNAzymes to the HeLa cells and trigger the enzymatic activity of DNAzymes resulting in exposure of the docking strand. Then, fix and permeabilize the cells. Finally, add the imager strands for DNA-PAINT. The location of labile metal ions will be converted to the position of docking strands inside the cells, which will give a snapshot of labile metal ions with super-resolution.

### Materials and Methods

All reagents for chemical synthesis were purchased from commercial sources and used as received. Sequences of oligonucleotide sequences used are shown in TABLE 1, where T′ and C′ are locked nucleic acids.

TABLE 1
Oligonucleotide sequences used in the experiments.

| | | SEQ ID |
|---|---|---|
| Oligonucleotides | Oligonucleotide sequences | NO |
| Dock-1-(SS)10 | 5′-/SS10//iSp18/CCT TCA ACA TAT CCT | 1 |
| | CTA/pyrene-dU/-3′ | |
| Imager1 ATTO488 | 5′-/5ATTO488N/AGA AGT AAT GTG GAA | 2 |
| | /3BHQ_1/-3′ | |
| Imager1 ATTO647N | 5′-/5ATTO647NN/AGA AGT AAT GTG GAA | 3 |
| | /3IAbRQSp/-3′ | |
| Imager2 ATTO647N | 5′-/5ATTO647NN/AAG AAG TAA AGG GAG | 4 |
| | /3IAbRQSp/-3′ | |
| 10-23 sub 3′NH2 | 5′-GTT GAA GGrA /rU/AATCATGC TT/NH2/-3′ | 5 |
| 10-23 sub 3′N3 | 5′-GTT GAA GGrA /rU/AATCATGC TT/N3/-3′ | 6 |
| 10-23ElnaTTC c18 caged | 5′-/DBCO/ TTG CAT GAT TAG GCT AGC TAC | 7 |
| 5′DBCO 3′NH2 | AAC/DEACM-G/AC CTT CAA CAT ATC CTC | |
| | T/C18/A AAA AAA T-/NH2/-3′ | |
| 10-23 lig LNA T′T′C′ | 5′- | 8 |
| caged A6T-3′NH2 | GTTGAAGGrArUAATCATGCTT/N3DBCO/TTG | |
| | CATGAT′T′AGGCTAGCTACAAC/DEACM- | |
| | G/ACC′TTCAACATATCCTCT/C18/A AAA AAA | |
| | T-NH2-3′ | |
| 10-23 lig LNA T′T′C′ | 5′- | 9 |
| caged A6T-3′N3 | GTTGAAGGrArUAATCATGCTT/N3DBCO/TTG | |
| | CATGAT′T′AGGCTAGCTACAAC/DEACM- | |
| | G/ACC′TTCAACATATCCTCT/C18/A AAA AAA | |
| | T-N3-3′ | |
| SS15-DBCO | 5′ /DBCO/ ATT TTT T/SS15/-3′ | 10 |
| 10-23 lig LNA T′T′C′ | 5′- | 11 |
| caged A6T-AT6-SS15 | GTTGAAGGrArUAATCATGCTT/N3DBCO/TTG | |
| | CATGAT′T′AGGCTAGCTACAAC/DEACM- | |
| | G/ACC′TTCAACATATCCTCT/C18/A AAA AAA | |
| | T-/N3DBCO/ATTTTTT/SS15/-3′ | |
| 10-23 lig LNA T′T′C′ | 5′- | 12 |
| caged A6T-3′ DBCO | GTTGAAGGrArUAATCATGCTT/N3DBCO/TTG | |
| | CATGAT′T′AGGCTAGCTACAAC/DEACM- | |
| | G/ACC′TTCAACATATCCTCT/C18/A AAA AAA | |
| | T-DBCO-3′ | |
| DNA DNAzyme | 5′- | 14 |
| | GTTGAAGGrArUAATCATGCTT/N3DBCO/TTG | |
| | CATGATTAGGCTAGCTACAAC/DEACM- | |
| | G/ACCTTCAACATATCCTCT-3′ | |

Synthesis of disulfide modified docking strand, and 10-23 LNA-DNAzyme: The Dock-1-(SS)10 was synthesized by using dithiol serinol phosphoramidite, and other common reagents (Glenresearch, USA) on an ABI 392 synthesizer and purified by HPLC. 10-23 sub 3′NH2 was synthesized by using 3′-amino-modifier serinol CPG, 2′-O-PivOM-5′-O-DMT-Adenosine (N-PAC) 3′-CEP, 2′-O-PivOM-5′-O-DMT-Uridine 3′-CEP and regular phosphoramidites. 10-23 sub 3′N₃ was synthesized through the postmodification of 10-23 sub 3′NH₂ with Azido-dPEG®₄-TFP ester. 10-23ElnaTTC c18 caged 5′DBCO 3′NH2 was synthesized by using spacer phosphoramidite 18, 5′-DBCO-TEG phosphoramidite, 3′-amino-modifier serinol CPG, and regular phosphoramidites. 10-23 lig LNA T′T′C′ caged A6T-3′NH2 was synthesized through the click reaction between 10-23 sub 3′N3 and 10-23ElnaTTC c18 caged 5′DBCO 3′NH2. 10-23 lig LNA T′T′C′ caged A6T-3′N3 was synthesized through the postmodification of 10-23 lig LNA T′T′C′ caged A6T-3′NH2 with Azido-dPEG®₄-TFP ester. 10-23 lig LNA T′T′C′ caged A6T-AT6-SS15 was synthesized through the click reaction between 10-23 lig LNA T′T′C′ caged A6T-3′N3 and SS15-DBCO. All the strands were purified by HPLC.

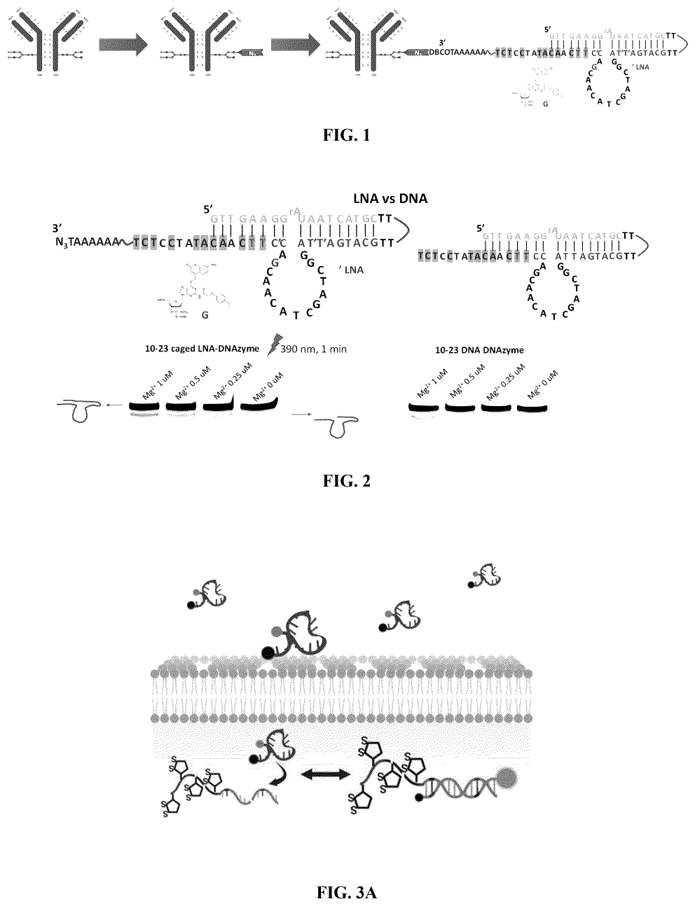

Synthesis of antibody-DNA conjugates: A schematic of the synthesis of DNA/DNAzyme-antibody conjugates is shown in FIG. 1.

Antibody-DNA conjugation was synthesized by click chemistry between azido-modified 2^{nd} antibody and DBCO-modified DNAs. Azido-modified 2^{nd} antibody could be obtained by using commercialized kit to selectively modify the 2^{nd} antibody. DBCO-modified DNA could be synthesized with corresponding monomers or through post-modification.

Activity tests of 10-23 LNA-modified DNAzymes and 10-23 DNAzymes: Activity tests of 10-23 LNA-modified DNAzymes and 10-23 DNAzymes were carried out in 50 mM Tris buffer with 150 mM NaCl (pH=7.4). 10-23 LNA-modified DNAzymes were activated with 390 nm laser irradiation for 1 min. Then, different concentrations of Mg²⁺ (0, 0.25, 0.5, and 1 mM) were added to test the cleavage efficiency by 20% denaturing PAGE gel after 1 h incubation with Mg²⁺.

Cell culture: HeLa cells were cultured in Dulbecco's Modification of Eagle's Medium (DMEM) supplemented with 10% Fetal Bovine Serum (FBS), 100 U/mL penicillin, and 100 mg/mL streptomycin (Cell Media Facility, UIUC), and were incubated at 37° C. in a humidified 5% CO₂ incubator. Before imaging, cells were transferred to 35 mm glass-bottomed culture dishes (MatTek Corp., Ashland, MA) and grown overnight to 70-90% confluency. Cells were transfected using different concentration of docking strand (Dock-1-(SS)10, 0.05 nM, 0.5 nM, 5 nM) in serum-free DMEM overnight. Cells were washed several times with PBS. The cells were permeabilized by using 3% paraformaldehyde and 0.1% glutaraldehyde for 15 min.

Super-resolution imaging of docking in HeLa cells: The permeabilized HeLa cells were in a high ionic-strength PBS-based buffer (1×PBS, 500 mM NaCl, 20 mM Na₂SO₃ and 1 mM Trolox, pH=7.4). Imager strands (Imager1 ATTO488) were added the HeLa cells at a final concentration of 20 nM. The images were obtained in a Nikon TIRF/N-STORM System using 50% laser intensity and 20 ms exposure time.

2D super-resolution imaging of microtubule, mitochondria, Mg²⁺ in HeLa cells: For Mg²⁺ imaging, Cells were transfected using disulfide-modified 10-23 LNA-DNAzyme (250 nM) in serum-free DMEM overnight. Cells for Mg²⁺ imaging were activated by 405 nm laser for 1 min and further incubated for 1 h. For microtubule and mitochondria imaging, cells were washed three times with PBS. Pre-extract the cells with prewarmed (37° C. for 10 min) pre-extraction buffer for 90 s. Remove the extraction buffer and fix the cells for 15 min in prewarmed enhanced microtubule fixative. Aspirate the fixative solution and reduce the sample with 1 mg/ml sodium borohydride for 7 min. Wash the chamber four times (1×20 s, 3×5 min) with 1×PBS at pH 7.4. Block and permeabilize the cell sample in blocking buffer for 90 min at RT. Dilute the primary antibody according to supplier instructions in antibody dilution buffer and incubate the sample at 4° C. overnight in a fridge on a rocking platform. Wash the sample three times for 5 min in 1×PBS. Dilute DNA-labeled secondary antibody (5-50 g/ml, 10 μg/mL) in antibody dilution buffer, and apply it to the sample for 60 min at RT on a rocking platform. Wash the sample three times for 5 min in 1×PBS. Add a target-specific imager solution (20 nM in PBS-based buffer (1×PBS, 500 mM NaCl, 20 mM Na₂SO₃ and 1 mM Trolox, pH=7.4)) to the sample. The images were obtained in a Nikon TIRF/N-STORM System using 50% laser intensity and 20 ms exposure time.

3D super-resolution imaging of microtubule, mitochondria, Mg²⁺ in HeLa cells: The 3D super-resolution imaging was performed in a similar way. The images were obtained in a lattice light sheet microscopy using 100% laser intensity, 20 ms exposure time.

Study of mitochondria-associated endoplasmic reticulum membranes' (MAMs') role during Mg²⁺ transportation can also be conducted.

### Results and Discussion

Engineering 10-23 LNA-DNAzyme for physiological Mg²⁺ sensing: LNA was introduced to 10-23 DNAzyme to push the cleavage could happen in physiological Mg²⁺ concentration because the previous report by Vester et al.¹² demonstrates the incorporation of LNA-type monomers into DNAzymes markedly increases RNA cleavage. The synthesized strands with modifications were characterized by MALDI-TOF. As shown in table 1, the observed MWs match the calculated MW, indicating the successful synthesis of 10-23 LNA-DNAzyme. After the successful preparation of 10-23 LNA-DNAzyme, the activity test was carried out to verify the engineered 10-23 LNA-DNAzyme could work in the physiological Mg²⁺ concentration (around 0.5 mM to 1.5 mM). The cleavage product band could be observed with 0.25 mM Mg²⁺ within 1 h, indicating that 10-23 LNA-DNAzyme could work in the physiological Mg²⁺ concentration. However, cleavage product of regular 10-23 DNAzyme could only be seen with 1 mM Mg²⁺, which make the 10-23 DNAzyme very challenging to work inside with local Mg concentration lower than 1 mM. Incorporation of LNA efficiently improves the cleavage of 10-23 DNAzyme, which will be adapted to engineer DNAzyme-PAIT for super-resolution Mg²⁺ sensing in single cells. These results are further shown in FIG. 2 and TABLE 2.

TABLE 2
MW of synthesized modified DNA with modifications
charactered by MALDI-TOF

| | Calculated | Observed |
|---|---|---|
| Oligonucleotides | MW (g/mol) | MW(g/mol) |
| 10-23 sub 3′N3 | 6686.3 | 6685.4 |
| 10-23ElnaTTC c18 caged | 17059.5 | 16737.4 |
| 5′DBCO 3′NH2 | | |
| 10-23 lig LNA T′T′C′ caged | 23807.8 | 23554.5 |
| A6T-3′NH2 | | |
| 10-23 lig LNA T′T′C′ caged | 24081.0 | 23667.2 |
| A6T-3′N3 | | |

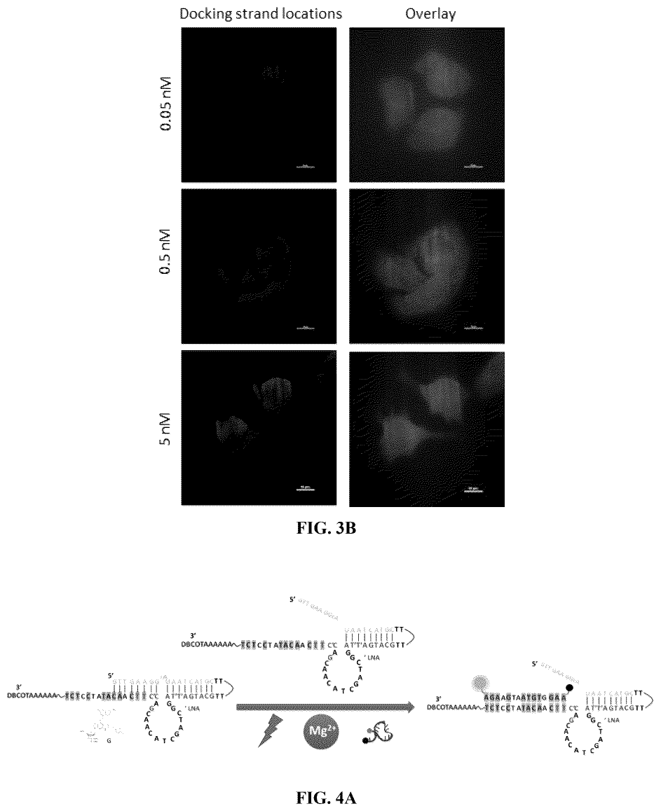

STORM imaging docking strand in cells: To demonstrate the fixed docking strands inside of HeLa cells could be specifically recognized by the imager strands and restored their super-resolution locations by fluorogenic DNA-PAINT. Different concentration of the docking strands (Dock-1-(SS)10, 0.05 nM, 0.5 nM, and 5 nM) were incubated with HeLa cells overnight, fixed and permeabilized for fluorogenic DNA-PAINT in HeLa cells by adding 20 nM imager strand (FIG. 3A). As shown in FIG. 3B, the location density of high concentration docking strand treated HeLa cells is higher than that of the low concentration docking strand, indicating that fluorogenic DNA-PAINT could be used as a quantitative tool to illustrate the concentration difference of docking strands. This is important for the future application of DNAzyme-PAINT to image the labile metal ions, which will convert the concentration and location information of liable metal ions into the concentration and location of the docking strand embedded in the DNAzyme binding arms.

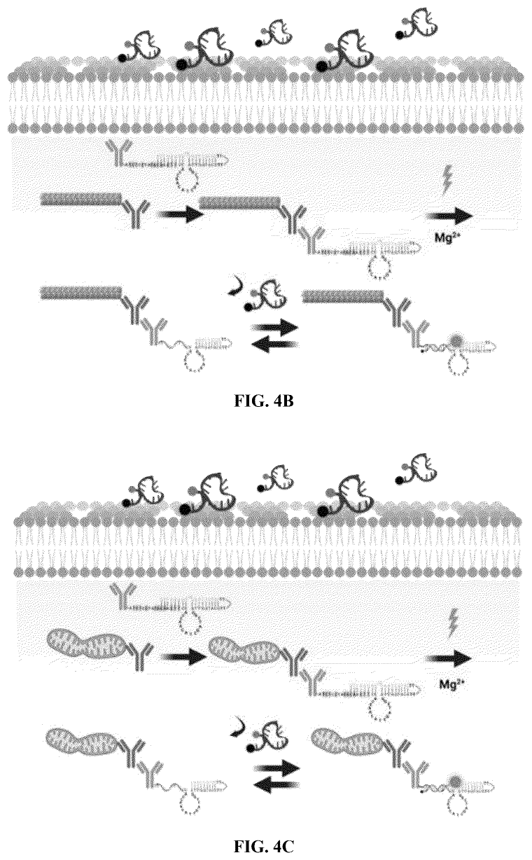

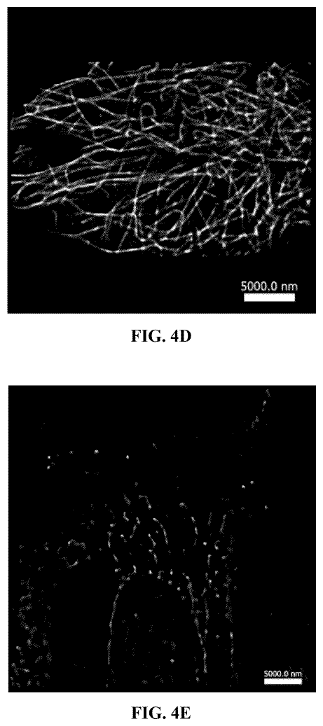

Super-resolution imaging of docking strand in cells (microtubules and mitochondria super-resolution imaging): After verification of fluorogenic DNA-PAINT in cells, fluorogenic 10-23 LNA-modified DNAzyme-PAINT (FIG. 4A) was engineered by embedding the docking strand in one of the binding arms of 10-23 LNA-modified DNAzyme. Once the cleavage reaction is triggered by light and magnesium, the imager probes will dock to the opening arm and generate blinking events to reconstruct the signal of interest. To demonstrate the feasibility of fluorogenic 10-23 LNA-modified DNAzyme-PAINT could work in the cells, the study used 10-23 LNA-modified DNAzyme-PAINT to paint the well-known structures inside the cells, such as microtubules and mitochondria. The fixed cells were incubated with primary antibody to microtubules and mitochondria, respectively. Then, corresponding 2^{nd} antibodies with 10-23 LNA-modified DNAzyme conjugation were added to the cells as shown in FIGS. 4A-4C. Only in the presence of 390 nm light and magnesium, 10-23 LNA-modified DNAzymes in the 2^{nd} antibodies could be activated, resulting in docking sites exposing. Thus, imager probes could dynamically bind to the docking sites, which will generate the blinking events for microtubule and mitochondrion structure reconstruction. As shown in FIGS. 4D-4E, both the microtubule and mitochondrion structure could be reconstructed, indicating the potential application of 10-23 LNA-modified DNAzyme-PAINT for labile Mg²⁺ biosensing in a single cell.

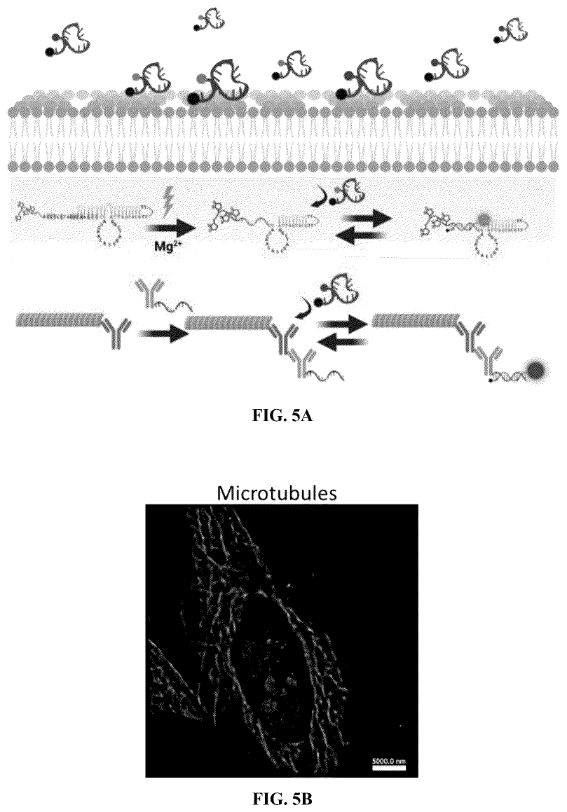

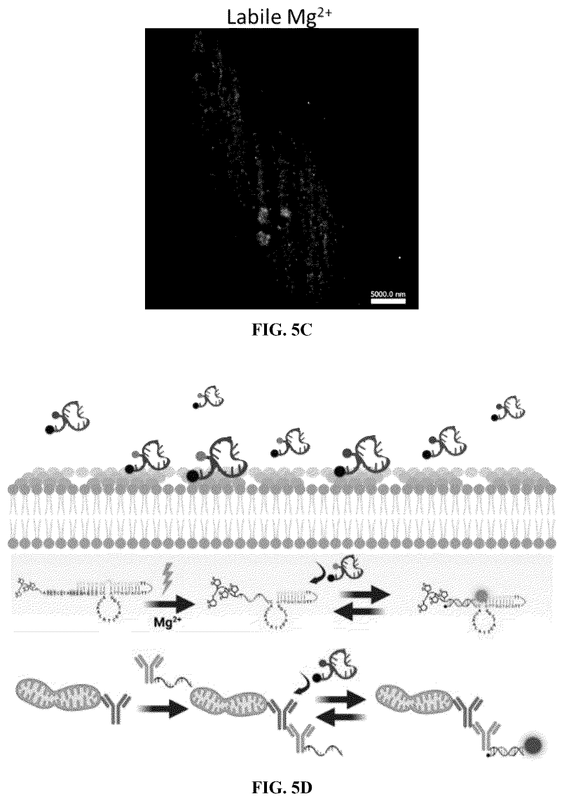

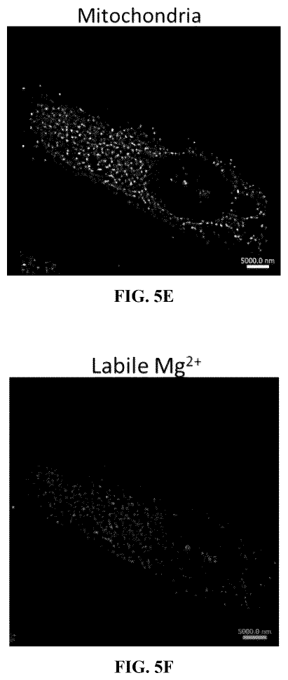

2D super-resolution imaging of Mg²⁺ in HeLa cells: To achieve the labile Mg²⁺ biosensing in a single cell, several challenges are faced for DNAzyme-based biosensor to achieve reliable outcomes including homogenous delivery of the biosensors, DNAzyme could function well with physiological metal ion cofactors, etc. Specifically for 10-23 DNAzyme, LNA was incorporated into the structure of 10-23 LNA-modified DNAzyme to realize 0.25 mM Mg²⁺ triggering substrate cleavage. It has been reported that disulfide modification is an efficient way to directly translate oligonucleotides into the cells. Thus introducing the disulfide modification at the terminal of 10-23 LNA-modified DNAzyme could achieve the homogenous delivery of the engineered biosensors for Mg²⁺ biosensing as shown in FIG. 5A and FIG. 5D. To locate the labile Mg²⁺ in cells, the study used a two color fluorogenic 10-23 LNA-modified DNAzyme-PAINT to indicate the both the abundance and the location of the labile Mg²⁺. Here the study used the one paint channel for microtubule or mitochondria, the other one for labile Mg²⁺ sensing. As shown in FIGS. 5B-5C, the locations of labile Mg²⁺ are not correlated with that of the microtubules. In contrast, the locations of labile Mg²⁺ are strongly correlated with that of the mitochondria, suggesting that Mg²⁺ might be transferred to or out of mitochondria. Recently, it has been reported that lactate acid mediated the transportation of Mg²⁺ from ER to mitochondria. This study achieved 2D imaging of Mg²⁺ by 10-23 LNA-modified DNAzyme-PAINT with TIRF/STORM. With the advanced of lattice light sheet microscope, this technique could be used for 3D imaging as well with 10-23 LNA-modified DNAzyme-PAINT.

3D super-resolution imaging of Mg²⁺ in HeLa cells: To realize 3D super-resolution imaging of Mg²⁺ in a single, a similar process was carried out as 2D super-resolution imaging. Very similar to the finding of 2D imaging results, the 3D locations of Mg²⁺ also correlate to mitochondria. This is the first time using DNAzyme to characterize the labile location in a single cell with high-resolution, which give us the clues to further study the Mg²⁺ transportation inside the cells. The location environment called mitochondria-associated membranes (MAMs) can play important role in Mg²⁺ transportation of between ER and mitochondria. Study of Mg²⁺ transportation in MAMs can also be conducted.

### CONCLUSIONS

To realize super-resolution imaging of the labile metal ion in cells, the study used DNAzyme-PAINT, which embeds the docking strands of fluorogenic DNA-PAINT in the DNAzyme binding arms. Once the cleavage of DNAzyme happens, the docking strand will expose and act as the dock of fluorogenic DNA-PAINT. By imaging the docking strand location and concentration, the concentration and location information of liable metal ions is known. To do DNAzyme-PAINT, first, the study verified that the docking strand location and concentration information can be presented in the fluorogenic DNA-PAINT. It was discovered that higher docking strand concentration lead to higher docking strand density in HeLa cells, which laid the foundation for DNAzyme-PAINT.

### Example 3: Spatial Metallomics: 3D Super-Resolution Imaging of Metal Ions in Cells Using Fluorogenic DNAzyme-PAINT

Metal ions play important roles in biological processes. Despite the importance, understanding of these metal ions is limited by a lack of general method to obtain molecules that are selective for metal ions and converting these molecules into sensors for simultaneous imaging metal ions with high spatial resolution. To overcome these limitations, a study was conducted which used intro selection to obtain DNAzymes that are selective for not only a wide variety of metal ions (e.g., Li⁺, Na⁺, and K⁺, Mg²⁺, Mn²⁺, Zn²⁺, and UO₂²⁺) but also different oxidation states of the same metal ions (e.g., Fe²⁺, Fe³⁺, Cu⁺, and Cu²⁺). The study converted these DNAzymes into fluorescent sensors using a catalytic beacon approach that allows using different fluorophores for simultaneous imaging metal ions in living cells, animal and human tissues. In addition, taking advantage of recent advances of super-resolution imaging technologies such as fluorogenic DNA-PAINT, this study developed a Fluorogenic DNAzyme-PAINT technique, which fuses Fluorogenic DNA-PAINT with DNAzyme catalysis for high-resolution imaging of labile metal ions. This Fluorogenic DNAzyme-PAINT technique, coupled with Lattice Light Sheet microscopy, allows 3D super-resolution mapping of intracellular metal ions, enhancing understanding of the vital role of labile metal ion pools in biological systems from mammalian cells to plants. These results provide a strong foundation toward spatial metallomics that will provide a comprehensive understanding of numerous metal ions in different biological processes.

### Example 4: DNAzyme-PAINT and Aptamer-PAINT

Metabolites and metal ions play essential roles in cellular processes, yet their dynamic and transient nature has posed a considerable challenge for cellular imaging. These molecules are often small and highly mobile, which makes them difficult to visualize using traditional fluorescence microscopy techniques. Super-resolution imaging methods, such as single-molecule localization microscopy (SMLM), have enabled visualization beyond the diffraction limit of light, yet they still face significant barriers when it comes to small, dynamic targets like metabolites and metal ions. DNA-PAINT (DNAbased Point Accumulation for Imaging in Nanoscale Topography) is one super-resolution technique that enhances imaging resolution by transiently binding fluorescently labeled oligonucleotides (imager probes) to complementary DNA sequences (docking strands) attached to the target of interest. Each binding event is registered as a “blink,” allowing for precise localization of the target. However, a major limitation of DNA-PAINT is the presence of free-floating imager probes that contribute to background fluorescence, which can obscure the signal from probes bound to the target, especially at the high probe concentrations necessary for faster imaging speeds. To minimize this background, researchers often use very low concentrations of probes or resort to techniques such as total internal reflection fluorescence (TIRF) microscopy, which restricts imaging to regions close to the coverslip, limiting the applicability for 3D imaging of whole cells or thicker samples. Additionally, existing methods to improve signal-to-noise ratios, such as the introduction of Forster Resonance Energy Transfer (FRET)-based systems, have their own trade-offs between signal brightness, imaging speed, and image quality. Other approaches, like those utilizing molecular beacons for their self-quenching properties, suffer from slow binding kinetics, making them incompatible with techniques like DNA-PAINT that require rapid binding and unbinding events to effectively “blink” and be localized. In summary, the challenge in 3D super resolution imaging of small and dynamic cellular components lies in achieving a balance between high signal-to-noise ratios, last imaging speeds, and maintaining high image resolutions for comprehensive and accurate depiction of cellular processes. Existing approaches have limitations in terms of probe design, speed of imaging, and specific detection of target molecules without excessive background interference, hindering the full realization of 3D super-resolution imaging's potential.

The invention presents two related technologies, Aptamer-PAINT and DNAzyme-PAINT, which enhance 30 super-resolution imaging, specifically targeting the visualization of small and dynamic entities such as metabolites and metal ions within cells—a task which is challenging due to their diminutive size and high mobility. The technical innovation lies in integrating the specificity of DNAzymes and aptamers, which are DNA molecules designed to bind particular molecules or ions, with the imaging technique DNA-PAINT. DNA-PAINT (Points Accumulation for Imaging in Nanoscale Topography) is a form of microscopy that relies on the transient binding of fluorescently labeled DNA strands to complementary sequences tethered to the target of interest. This tethering causes the fluorescent probes to “blink”, and these blinking events are localized with high precision to generate an image at nanometer resolution. The fluorogenic DNA-PAINT uses specially designed fluorescent probes (imager probes) conjugated with both a fluorophore and a quencher. These probes are designed to be dark (non-fluorescent) when unbound in solution and become brightly fluorescent upon binding to their target DNA strands within the cell. This minimizes background fluorescence, which has traditionally been a major limitation in DNA-PAINT imaging, leading to an improvement in imaging speed, a reduction in imaging-related noise, and the ability to conduct 30 super-resolution imaging in cells without optical sectioning. The manuscript demonstrates a 26-fold increase in imaging speed compared to traditional DNA-PAINT thanks to the optimized fluorogenic imager probes which allow a greater probe concentration and use faster camera frame rates, reducing imaging time from hours to minutes. It also successfully provides dual color imaging with minimal cross-talk between probes, which enables simultaneous visualization of two cellular structures. This approach advances understanding of cell biology by allowing for the detailed visualization of cellular processes involving metabolites and metal ions without the drawbacks of slow speeds and high background interference characterizing older imaging methods.

Enhanced imaging speed: Aptamer-PAINT and DNAzyme-PAINT technologies significantly increase imaging speed, offering a 26-fold improvement over regular DNA-PAINT. Traditional DNA-PAINT imaging, as mentioned in the document, can acquire super resolution images in minutes instead of hours, but still required TIRF microscopy to maintain low background. Aptamer-PAINT and DNAzyme-PAINT offer a faster alternative without this limitation.

Reduced background fluorescence: The invention notably reduces background fluorescence associated with unbound fluorophores, which has been a substantial limitation in conventional DNA-PAINT. The technology uses self-quenching, kinetics optimized probe designs that significantly reduce the brightness of diffusing unbound probes compared to the bound state, facilitating clearer imaging with less interference.

3D super-resolution imaging without optical sectioning: The capability for 3D super resolution DNA-PAINT imaging without the need for optical sectioning sets this technology apart from existing approaches like regular DNA-PAINT and STORM (Stochastic Optical Reconstruction Microscopy), which often require TIRF or spinning disk confocal microscopy hence limiting the observation volume to regions close to the coverslip.

Capability for multicolor imaging: The technology allows for simultaneous multicolor imaging using orthogonal sets of imager probes and docking strands labeled with different fluorophores, facilitating the observation of multiple targets at once. This multicolor capability is essential for detailed cellular studies and surpasses the sequential multiplex imaging in methods such as Exchange-PAINT, which is more time-consuming.

High fluorogenicity and fast binding kinetics: The fluorogenic imager probes paired with partially mismatched docking strands are designed to achieve both high fluorogenicity (bright fluorescence upon binding) and fast binding kinetics, necessary for rapid image acquisition. This design overcomes limits of other approaches like FRET-based DNA-PAINT that can have reduced image quality due to FRET inefficiency and fluorophore crosstalk, and molecular beacons, which have fluorogenicity but are not compatible with the required kinetics for DNA-PAINT.

### EXAMPLE ASPECTS

Example 1: A method for spatially identifying a target molecule in a cell, the method comprising: a) providing to the cell a modified DNAzyme or aptamer comprising a docking site; b) providing to the cell an imaging probe comprising a detectable signal and a sequence complimentary to the docking site of the modified DNAzyme or aptamer; c) exposing the cell or tissue sample to a light source; wherein the imaging probe binds to the docking site in the presence of the target molecule and the light source; and d) imaging the cell, thereby allowing for spatial identification of the target molecule.

Example 2: The method according to any examples herein, particularly Example 1, wherein the modified DNAzyme or aptamer comprises one or more locked nucleic acids (LNAs).

Example 3: The method according to any examples herein, particularly Examples 1 or 2, wherein the modified DNAzyme or aptamer further comprises an antibody conjugated to the modified DNAzyme or aptamer; and further wherein the antibody binds to a naturally occurring antibody presented on a structure within the cell.

Example 4: The method according to any examples herein, particularly Example 3, wherein the structure within the cell is a microtubule or mitochondrion.

Example 5: The method according to any examples herein, particularly Examples 3 or 4, wherein the antibody binds to the structure within the cell.

Example 6: The method according to any examples herein, particularly Examples 1-5, wherein the modified DNAzyme or aptamer undergoes a cleavage event in the presence of the target molecule, wherein said cleavage event exposes single stranded nucleic acid which can then hybridize with the imaging probe.

Example 7: The method according to any examples herein, particularly Examples 1-6, wherein the cell is within a tissue.

Example 8: The method according to any examples herein, particularly Examples 1-7, wherein the cell is in vitro, in silico, in situ or in vivo.

Example 9: The method according to any examples herein, particularly Examples 1-8, wherein the cell is mammalian.

Example 10: The method according to any examples herein, particularly Example 9, wherein the cell is human.

Example 11: The method according to any examples herein, particularly Examples 1-8, wherein the cell is a plant cell.

Example 12: The method according to any examples herein, particularly Examples 1-11, wherein the detectable signal is a fluorophore or a fluorescent dye.

Example 13: The method according to any examples herein, particularly Examples 1-12, wherein the imaging probe comprises 80% identity or more to any one of SEQ ID NOs: 2-4.

Example 14: The method according to any examples herein, particularly Example 13, wherein the imaging probe comprises 90% identity or more to any one of SEQ ID NOs: 2-4.

Example 15: The method according to any examples herein, particularly Example 14, wherein the imaging probe comprises any one of SEQ ID NOs: 2-4.

Example 16: The method according to any examples herein, particularly Examples 1-15, wherein the modified DNAzyme comprises 80% similarity or more to any one of SEQ ID NOs: 5-9 or SEQ ID NOs: 11-12.

Example 17: The method according to any examples herein, particularly Example 16, wherein the modified DNAzyme comprises 90% similarity or more to any one of SEQ ID NOs: 5-9 or SEQ ID NOs: 11-12.

Example 18: The method according to any examples herein, particularly Example 17, wherein the modified DNAzyme comprises any one of SEQ ID NOs: 5-9 or SEQ ID NOs: 11-12.

Example 19: The method according to any examples herein, particularly Examples 1-18, wherein the light source irradiates visible light.

Example 20: The method according to any examples herein, particularly Example 19, wherein the light source has a wavelength of from about 500 nm to about 350 nm.

Example 21: The method according to any examples herein, particularly Examples 1-20, wherein the cell is irradiated for from about 10 seconds to about 2 minutes.

Example 22: The method according to any examples herein, particularly Examples 1-21, wherein step a) further comprises incubating the cell with the modified DNAzyme or aptamer to allow the cell to uptake the modified DNAzyme or aptamer.

Example 23: The method according to any examples herein, particularly Example 22, wherein the modified DNAzyme or aptamer is incubated from about 30 minutes to about 72 hours.

Example 24: The method according to any examples herein, particularly Examples 1-23, wherein the target molecule is a metal ion.

Example 25: The method according to any examples herein, particularly Example 24, wherein the metal ion is one or more of Li⁺, Na⁺, K⁺, Mg²⁺, Mn²⁺, Zn²⁺, UO₂²⁺, Fe²⁺, Fe³⁺, Cu⁺, or Cu²⁺.

Example 26: The method according to any examples herein, particularly Example 25, wherein the metal ion is Mg²⁺.

Example 27: The method according to any examples herein, particularly Examples 1-26, further comprising, after step c) and before step d), washing the cell with a buffer.

Example 28: The method according to any examples herein, particularly Examples 1-27, wherein the cell is imaged using 3D imaging.

Example 29: The method according to any examples herein, particularly Example 28, wherein the imaging is done using a super-resolution microscope.

Example 30: The method according to any examples herein, particularly Example 29, wherein the microscope comprises stimulated emission depletion (STED) microscopy, structured illumination microscopy (SIM), and stochastic optical reconstruction microscopy (STORM)/photoactivation localization microscopy (PALM).

Example 31: The method according to any examples herein, particularly Examples 1-30, wherein the method is used to multiplex more than one target molecule at a time.

Example 32: The method according to any examples herein, particularly Example 31, wherein at least one target molecule is a metal ion and one is not.

Example 33: The method according to any examples herein, particularly Example 31, wherein two different metal ions are detected simultaneously.

Example 34: The method according to any examples herein, particularly Examples 31-33, wherein two or more different modified DNAzymes are used simultaneously.

Example 35: The method according to any examples herein, particularly Example 34, wherein the two or more different modified DNAzymes use different docking sites.

Example 36: The method according to any examples herein, particularly Examples 34 or 35, wherein the two or more different modified DNAzymes use the same or different imaging probes.

Example 37: The method according to any examples herein, particularly Examples 1-36, wherein the method is used to quantify the target molecule in the cell.

Example 38: The method according to any examples herein, particularly Example 1-37, wherein software is used to analyze data.

Example 39: The method according to any examples herein, particularly Example 38, wherein said software package is Picasso.

Example 40: A modified DNAzyme comprising 80% similarity or more to any one of SEQ ID NOs: 5-9 or SEQ ID NOs: 11-12.

Example 41: The modified DNAzyme according to any examples herein, particularly Example 40, wherein the modified DNAzyme comprises 90% similarity or more to any one of SEQ ID NOs: 5-9 or SEQ ID NOs: 11-12.

Example 42: The modified DNAzyme according to any examples herein, particularly Example 42, wherein the modified DNAzyme comprises SEQ ID NOs: 5-9 or SEQ ID NOs: 11-12.

Example 43: The modified DNAzyme according to any examples herein, particularly Examples 40-43, further comprising an antibody conjugated to the modified DNAzyme.

Example 44: The modified DNAzyme according to any examples herein, particularly Example 43, wherein the antibody is a primary antibody to a microtubule or a mitochondrion.

Example 45: A cell comprising the modified DNAzyme according to any examples herein, particularly Examples 40-44.

Example 46: A DNAzyme sensor comprising: a substrate strand comprising a cleavage site, wherein the cleavage site is uncleaved when magnesium (Mg²⁺) is not present; and an enzyme strand at least partially complementary to the substrate strand and comprising a catalytic loop; wherein the catalytic loop is capable of cleaving the substrate strand at the cleavage site in the presence of Mg²⁺, wherein said cleavage provides a detectable signal; and wherein the catalytic loop comprises SEQ ID NO: 13 or a variant thereof.

Example 46: The DNAzyme sensor of any examples herein, particularly Example 45, wherein the cleavage site comprises from 1 RNA base to 5 RNA bases.

Example 47: The DNAzyme sensor of any examples herein, particularly Example 45, wherein the substrate strand and the enzyme strand are linked together.

Example 48: The DNAzyme sensor of any examples herein, particularly Example 47, wherein the DNAzyme sensor comprises about 80% similarity or more to any one of SEQ ID NOs: 5-9 or SEQ ID NOs: 11-12.

Example 49: The DNAzyme sensor of any examples herein, particularly Example 45, further comprising an antibody conjugated to the DNAzyme sensor; and wherein the antibody binds to a structure within the cell.

Example 50: The DNAzyme sensor of any examples herein, particularly Example 49, wherein the antibody binds to a microtubule or a mitochondrion.

Example 51: The DNAzyme sensor of any examples herein, particularly Example 45, wherein the detectable signal is a fluorophore or fluorescent dye; wherein the fluorophore or fluorescent dye is conjugated to a first end of the substrate strand; and wherein a quencher is conjugated to a complementary end of the enzyme strand or a second end of the substrate strand.

Example 52: The DNAzyme sensor of any examples herein, particularly Example 45, wherein the DNAzyme sensor comprises a docking site that is at least partially complementary to the substrate strand; and wherein, upon cleavage of the substrate strand, the docking site is at least partially exposed.

Example 53: The DNAzyme sensor of any examples herein, particularly Example 52, wherein cleavage of the substrate strand decreases melting temperature of the DNAzyme sensor, thereby causing the cleaved portion of the substrate strand to at least partially disassociate from the docking site.

Example 54: A kit comprising the DNAzyme sensor of any examples herein, particularly Example 52 and an imaging probe; wherein the imaging probe comprises the detectable signal and a sequence at least partially complementary to the docking site; and wherein, upon cleavage of the substrate strand in the presence of Mg²⁺, the imaging probe binds to the docking site on the DNAzyme sensor, thereby providing a detectable signal.

Example 55: The kit of any examples herein, particularly Example 54, wherein the detectable signal is a fluorophore or fluorescent dye conjugated to the imaging probe.

Example 56: The kit of any examples herein, particularly Example 54, wherein the imaging probe comprises about 80% similarity or more to any one of SEQ ID NOs: 2-4.

Example 57: A method for spatially identifying a target molecule in a cell, the method comprising: a) providing to the cell a DNAzyme sensor comprising: i) a substrate strand comprising a cleavage site, wherein the cleavage site is initially uncleaved; ii) an enzyme strand at least partially complementary to the substrate strand and comprising a catalytic loop; iii) a docking site at least partially complementary to the substrate strand; wherein the catalytic loop is capable of cleaving the substrate strand at the cleavage site in the presence of a target molecule; b) providing to the cell an imaging probe comprising a detectable signal and a sequence at least partially complementary to the docking site of the DNAzyme sensor, wherein, upon cleavage of the substrate strand in the presence of the target molecule, the imaging probe binds to the docking site; and c) imaging the cell, thereby allowing for spatial identification of the target molecule.

Example 58: The method of any examples herein, particularly Example 57, wherein the DNAzyme sensor further comprises an antibody conjugated to the DNAzyme sensor; and wherein the antibody binds to a structure within the cell.

Example 59: The method of any examples herein, particularly Example 58, wherein the structure within the cell is a microtubule or mitochondrion.

Example 60: The method of any examples herein, particularly Example 57, wherein the detectable signal is a fluorophore or a fluorescent dye conjugated to the imaging probe.

Example 61: The method of any examples herein, particularly Example 57, wherein step a) further comprises irradiating the cell with a light source to activate cleavage of the substrate strand.

Example 62: The method of any examples herein, particularly Example 57, wherein the target molecule is a metal ion selected from one or more of Li⁺, Na⁺, K⁺, Mg²⁺, Mn²⁺, Zn²⁺, UO₂²⁺, Fe²⁺, Fe³⁺, Cu⁺, or Cu²⁺.

Example 63: The method of any examples herein, particularly Example 57, wherein step a) further comprises providing to the cell two or more DNAzyme sensors, wherein the catalytic loop of each DNAzyme sensor is capable of cleaving each substrate strand at its cleavage site in the presence of a different target molecule; and wherein the method is used to multiplex more than one target molecule at a time.

Example 64: The method of any examples herein, particularly Example 63, wherein at least one target molecule is a metal ion.

The following patents, applications and publications as listed below and throughout this document are hereby incorporated by reference in their entirety herein.

### REFERENCE LIST

1. Dean, K. M.; et al., Visualizing metal ions in cells: An overview of analytical techniques, approaches, and probes. Biochimica et Biophysica Acta (BBA)—Molecular Cell Research 2012, 1823 (9), 1406-1415.
2. Fleming, R. E.; Ponka, P., Iron overload in human disease. N Engl J Med 2012, 366 (4), 348-59.
3. Prasad, A. S., Zinc deficiency in patients with sickle cell disease. Am J Clin Nutr 2002, 75 (2), 181-2.
4. Domaille, D. W.; et al., Synthetic fluorescent sensors for studying the cell biology of metals. Nature chemical biology 2008, 4 (3), 168-175.
5. Tottey, S.; et al., Protein-folding location can regulate manganese-binding versus copper- or zinc-binding. Nature 2008, 455 (7216), 1138-1142.
6. Jungmann, R.; et al., Single-Molecule Kinetics and Super-Resolution Microscopy by Fluorescence Imaging of Transient Binding on DNA Origami. Nano Letters 2010, 10 (11), 4756-4761.
7. Auer, A.; et al., Fast, Background-Free DNA-PAINT Imaging Using FRET-Based Probes. Nano Letters 2017, 17 (10), 6428-6434.
8. Schnitzbauer, J.; et al., Super-resolution microscopy with DNA-PAINT. Nature Protocols 2017, 12 (6), 1198-1228.
9. Chen, Y.; et al., Empowering single-molecule analysis with self-assembled DNA nanostructures. Matter 2021, 4 (10), 3121-3145.
10. van Wee, R.; et al., Completing the canvas: advances and challenges for DNA-PAINT super-resolution imaging. Trends in Biochemical Sciences 2021, 46 (11), 918-930.
11. Chung, K. K. H.; et al., Fluorogenic DNA-PAINT for faster, low-background super-resolution imaging. Nature Methods 2022.
12. Vester, B.; et al., LNAzymes: Incorporation of LNA-Type Monomers into DNAzymes Markedly Increases RNA Cleavage. Journal of the American Chemical Society 2002, 124 (46), 13682-13683.
13. U.S. Pat. No. 10,753,892 B2
14. US 2018/236107 A1
15. U.S. Pat. No. 11,591,574 B2
16. WO 2022/121276 A1
17. U.S. Pat. No. 9,751,910 B2

What is claimed is:

1. A DNAzyme sensor comprising:
a substrate strand comprising a cleavage site, wherein the cleavage site is uncleaved when magnesium (Mg²⁺) is not present; and
an enzyme strand at least partially complementary to the substrate strand and comprising a catalytic loop;
wherein the catalytic loop is capable of cleaving the substrate strand at the cleavage site in the presence of Mg²⁺, wherein said cleavage provides a detectable signal; and
wherein the catalytic loop comprises SEQ ID NO: 13 or a variant thereof.

2. The DNAzyme sensor of claim 1, wherein the cleavage site comprises from 1 RNA base to 5 RNA bases.

3. The DNAzyme sensor of claim 1, wherein the substrate strand and the enzyme strand are linked together.

4. The DNAzyme sensor of claim 3, wherein the DNAzyme sensor comprises about 80% similarity or more to any one of SEQ ID NOs: 5-9 or SEQ ID NOs: 11-12.

5. The DNAzyme sensor of claim 1, further comprising an antibody conjugated to the DNAzyme sensor; and
wherein the antibody binds to a structure within the cell.

6. The DNAzyme sensor of claim 5, wherein the antibody binds to a microtubule or a mitochondrion.

7. The DNAzyme sensor of claim 1, wherein the detectable signal is a fluorophore or fluorescent dye;
wherein the fluorophore or fluorescent dye is conjugated to a first end of the substrate strand; and
wherein a quencher is conjugated to a complementary end of the enzyme strand or a second end of the substrate strand.

8. The DNAzyme sensor of claim 1, wherein the DNAzyme sensor comprises a docking site that is at least partially complementary to the substrate strand; and
wherein, upon cleavage of the substrate strand, the docking site is at least partially exposed.

9. The DNAzyme sensor of claim 8, wherein cleavage of the substrate strand decreases melting temperature of the DNAzyme sensor, thereby causing the cleaved portion of the substrate strand to at least partially disassociate from the docking site.

10. A kit comprising the DNAzyme sensor of claim 8 and an imaging probe;
wherein the imaging probe comprises the detectable signal and a sequence at least partially complementary to the docking site; and
wherein, upon cleavage of the substrate strand in the presence of Mg²⁺, the imaging probe binds to the docking site on the DNAzyme sensor, thereby providing a detectable signal.

11. The kit of claim 10, wherein the detectable signal is a fluorophore or fluorescent dye conjugated to the imaging probe.

12. The kit of claim 11, wherein the imaging probe comprises about 80% similarity or more to any one of SEQ ID NOs: 2-4.

13. A method for spatially identifying a target molecule in a cell, the method comprising:
a) providing to the cell a DNAzyme sensor comprising:
i) a substrate strand comprising a cleavage site, wherein the cleavage site is initially uncleaved;
ii) an enzyme strand at least partially complementary to the substrate strand and comprising a catalytic loop;
iii) a docking site at least partially complementary to the substrate strand;
wherein the catalytic loop is capable of cleaving the substrate strand at the cleavage site in the presence of a target molecule;
b) providing to the cell an imaging probe comprising a detectable signal and a sequence at least partially complementary to the docking site of the DNAzyme sensor, wherein, upon cleavage of the substrate strand in the presence of the target molecule, the imaging probe binds to the docking site; and
c) imaging the cell, thereby allowing for spatial identification of the target molecule.

14. The method of claim 13, wherein the DNAzyme sensor further comprises an antibody conjugated to the DNAzyme sensor; and
wherein the antibody binds to a structure within the cell.

15. The method of claim 14, wherein the structure within the cell is a microtubule or mitochondrion.

16. The method of claim 13, wherein the detectable signal is a fluorophore or a fluorescent dye conjugated to the imaging probe.

17. The method of claim 13, wherein step a) further comprises irradiating the cell with a light source to activate cleavage of the substrate strand.

18. The method of claim 13, wherein the target molecule is a metal ion selected from one or more of Li⁺, Na⁺, K⁺, Mg²⁺, Mn²⁺, Zn²⁺, UO₂²⁺, Fe²⁺, Fe³⁺, Cu⁺, or Cu²⁺.

19. The method of claim 13, wherein step a) further comprises providing to the cell two or more DNAzyme sensors, wherein the catalytic loop of each DNAzyme sensor is capable of cleaving each substrate strand at its cleavage site in the presence of a different target molecule; and
wherein the method is used to multiplex more than one target molecule at a time.

20. The method of claim 19, wherein at least one target molecule is a metal ion.


SEQUENCE LISTING (13 sequences)

SEQ ID NO: 1 - DNA, 18 bp, synthetic construct
  misc_feature 1..18: note = Description of sequence: Oligonucleotide
  source 1..18: mol_type = other DNA
  modified_base 1: mod_base = OTHER; note = cytosine-(SH-R-SH)10-C18-cytosine
  modified_base 18: mod_base = OTHER; note = pyrene-d-uracil
cttcaacatatcctctat

SEQ ID NO: 2 - DNA, 15 bp, synthetic construct
  misc_feature 1..15: note = Description of sequence: Oligonucleotide
  source 1..15: mol_type = other DNA
  modified_base 1: mod_base = OTHER; note = adenine modified with fluorescent dye, name of dye: 5' ATTO 488 (NHS Ester)
  modified_base 15: mod_base = OTHER; note = adenine modified with quencher, name of quencher: 3' Black Hole Quencher-1
agaagtaatgtggaa

SEQ ID NO: 3 - DNA, 15 bp, synthetic construct
  misc_feature 1..15: note = Description of sequence: Oligonucleotide
  source 1..15: mol_type = other DNA
  modified_base 1: mod_base = OTHER; note = adenine modified with fluorescent dye, name of dye: 5' ATTO 647N (NHS Ester)
  modified_base 15: mod_base = OTHER; note = adenine modified with quencher, name of quencher: 3' Iowa Black RQ
agaagtaatgtggaa

SEQ ID NO: 4 - DNA, 15 bp, synthetic construct
  misc_feature 1..15: note = Description of sequence: Oligonucleotide
  source 1..15: mol_type = other DNA
  modified_base 1: mod_base = OTHER; note = adenine modified with fluorescent dye, name of dye: 5' ATTO 647N (NHS Ester)
  modified_base 15: mod_base = OTHER; note = guanine modified with quencher, name of quencher: 3' Iowa Black RQ
aagaagtaaagggag

SEQ ID NO: 5 - DNA, 20 bp, synthetic construct
  misc_feature 1..20: note = Description of sequence: Oligonucleotide
  source 1..20: mol_type = other DNA
  modified_base 10: mod_base = OTHER; note = Uracil
  misc_feature 1..7: note = DNA
  misc_feature 7..8: note = RNA
  misc_feature 9..20: note = DNA
  modified_base 20: mod_base = OTHER; note = thymine-NH2
gttgaaggataatcatgctt

SEQ ID NO: 6 - DNA, 20 bp, synthetic construct
  misc_feature 1..20: note = Description of sequence: Oligonucleotide
  source 1..20: mol_type = other DNA
  modified_base 10: mod_base = OTHER; note = Uracil
  misc_feature 1..7: note = DNA
  misc_feature 7..8: note = RNA
  misc_feature 9..20: note = DNA
  modified_base 20: mod_base = OTHER; note = thymine-azido
gttgaaggataatcatgctt

SEQ ID NO: 7 - DNA, 50 bp, synthetic construct
  misc_feature 1..50: note = Description of sequence: Oligonucleotide
  source 1..50: mol_type = other DNA
  modified_base 1: mod_base = OTHER; note = Dibenzocyclooctyne-thymine
  modified_base 25: mod_base = OTHER; note = 5'-Dimethoxytrityl-N2-(4-isopropylphenoxyacetyl)-O6 -[[7-(diethylamino)coumarin-4-yl]-methyl]-2'-deoxyGuanosin e, 3'-[(2-cyanoethyl)-(N,N-diisopropyl)]-phosphoramidite
  modified_base 43: mod_base = OTHER; note = thymine-C18-adenine
  modified_base 50: mod_base = OTHER; note = thymine-NH2
ttgcatgattaggctagctacaacgaccttcaacatatcctctaaaaaat

SEQ ID NO: 8 - DNA, 69 bp, synthetic construct
  misc_feature 1..69: note = Description of sequence: Oligonucleotide
  source 1..69: mol_type = other DNA
  modified_base 10: mod_base = OTHER; note = Uracil
  misc_feature 1..7: note = DNA
  misc_feature 7..8: note = RNA
  misc_feature 9..69: note = DNA
  modified_base 20: mod_base = OTHER; note = thymine-azido-dibenzocyclooctyne-thymine
  modified_base 44: mod_base = OTHER; note = 5'-Dimethoxytrityl-N2-(4-isopropylphenoxyacetyl)-O6 -[[7-(diethylamino)coumarin-4-yl]-methyl]-2'-deoxyGuanosin e, 3'-[(2-cyanoethyl)-(N,N-diisopropyl)]-phosphoramidite
  modified_base 62: mod_base = OTHER; note = thymine-C18-adenine
  modified_base 69: mod_base = OTHER; note = thymine-NH2
gttgaaggataatcatgctttgcatgattaggctagctacaacgaccttcaacatatcctctaaaaaat

SEQ ID NO: 9 - DNA, 69 bp, synthetic construct; identical to SEQ ID NO: 8
  misc_feature 1..69: note = Description of sequence: Oligonucleotide
  source 1..69: mol_type = other DNA
  modified_base 10: mod_base = OTHER; note = Uracil
  misc_feature 1..7: note = DNA
  misc_feature 7..8: note = RNA
  misc_feature 9..69: note = DNA
  modified_base 20: mod_base = OTHER; note = thymine-azido-dibenzocyclooctyne-thymine
  modified_base 44: mod_base = OTHER; note = 5'-Dimethoxytrityl-N2-(4-isopropylphenoxyacetyl)-O6 -[[7-(diethylamino)coumarin-4-yl]-methyl]-2'-deoxyGuanosin e, 3'-[(2-cyanoethyl)-(N,N-diisopropyl)]-phosphoramidite
  modified_base 62: mod_base = OTHER; note = thymine-C18-adenine
  modified_base 69: mod_base = OTHER; note = thymine-NH2

SEQ ID NO: 10 - skipped

SEQ ID NO: 11 - DNA, 75 bp, synthetic construct
  misc_feature 1..75: note = Description of sequence: Oligonucleotide
  source 1..75: mol_type = other DNA
  modified_base 10: mod_base = OTHER; note = Uracil
  misc_feature 1..7: note = DNA
  misc_feature 7..8: note = RNA
  misc_feature 9..75: note = DNA
  modified_base 20: mod_base = OTHER; note = thymine-azido-dibenzocyclooctyne-thymine
  modified_base 44: mod_base = OTHER; note = 5'-Dimethoxytrityl-N2-(4-isopropylphenoxyacetyl)-O6 -[[7-(diethylamino)coumarin-4-yl]-methyl]-2'-deoxyGuanosin e, 3'-[(2-cyanoethyl)-(N,N-diisopropyl)]-phosphoramidite
  modified_base 62: mod_base = OTHER; note = thymine-C18-adenine
  modified_base 69: mod_base = OTHER; note = thymine-azido-dibenzocyclooctyne-adenine
  modified_base 75: mod_base = OTHER; note = thymine-(SH-R-SH)15
gttgaaggataatcatgctttgcatgattaggctagctacaacgaccttcaacatatcctctaaaaaattttttt

SEQ ID NO: 12 - DNA, 69 bp, synthetic construct; identical to SEQ ID NO: 8
  misc_feature 1..69: note = Description of sequence: Oligonucleotide
  source 1..69: mol_type = other DNA
  modified_base 10: mod_base = OTHER; note = Uracil
  misc_feature 1..7: note = DNA
  misc_feature 7..8: note = RNA
  misc_feature 9..69: note = DNA
  modified_base 20: mod_base = OTHER; note = thymine-azido-dibenzocyclooctyne-thymine
  modified_base 44: mod_base = OTHER; note = 5'-Dimethoxytrityl-N2-(4-isopropylphenoxyacetyl)-O6 -[[7-(diethylamino)coumarin-4-yl]-methyl]-2'-deoxyGuanosin e, 3'-[(2-cyanoethyl)-(N,N-diisopropyl)]-phosphoramidite
  modified_base 62: mod_base = OTHER; note = thymine-C18-adenine
  modified_base 69: mod_base = OTHER; note = thymine-dibenzocyclooctyne

SEQ ID NO: 13 - DNA, 15 bp, synthetic construct
  misc_feature 1..15: note = Description of sequence: oligionucleotide
  source 1..15: mol_type = other DNA
ggctagctacaacga